(19) United States
(12) Patent Application Publication
(10) Pub. No.: US 2005/0154426 A1
(43) Pub. Date: Jul. 14, 2005

(54) Method and system for providing therapy for neuropsychiatric and neurological disorders utilizing transcranical magnetic stimulation and pulsed electrical vagus nerve(s) stimulation

(76) Inventors: Birinder R. Boveja, Milwaukee, WI (US); Angely Widhany, Milwaukee, WI (US)

Correspondence Address: BIRINDER R. BOVEJA & ANGELY WIDHANY, P. O. BOX 210095, MILWAUKEE, WI, 53221, US

(21) Appl. No.: 11/074,130

(22) Filed: Mar. 7, 2005

Related U.S. Application Data
(63) Continuation-in-part of application No. 10/196,533, filed on Jul. 16, 2002.
Application No. 10/196,533 is a continuation of application No. 10/142,298, filed on May 9, 2002.

(51) Int. Cl. A61N 1/18

(52) U.S. Cl. 607/45

(57) ABSTRACT

A method and system of providing therapy or alleviating the symptoms of neuropsychiatric disorders and cognitive impairments comprises, providing repetitive transcranial magnetic stimulation (rTMS) to the brain and pulsed electrical stimulation to the vagus nerve(s) for afferent neuromodulation. These neuropsychiatric disorders and cognitive impairments include depression, bipolar depression, anxiety disorders, obsessive-compulsive disorders, schizophrenia, borderline personality disorders, sleep disorders, learning difficulties, memory impairments and the like. rTMS is provided to the brain via external coil which may be either circular in shape or figure-eight shaped. The frequency of TMS may be 1 Hz, 5 Hz, 20 Hz, or 60 Hz. RTMS may be provided via square pulses or sine wave pulses. Pulsed electrical stimulation to the vagus nerve(s) may be provided continuously in ON-OFF repeating cycles. The two stimulation therapies may be given in any order, any combination, or any sequence as determined by the physician. The two stimulation therapies may also be used with or without pharmaceutical therapy. Pulsed electrical vagus nerve stimulation (VNS) may be provided using an implanted pulse generator (IPG) or an external stimulator used in conjunction with an implanted stimulus-receiver. In one aspect of the invention the pulse generator system may comprise communication capabilities for networking over a wide area network, for remote interrogation and programming.

This application is a continuation of application Ser. No. 10/196,533 filed Jul. 16, 2002, entitled “METHOD AND SYSTEM FOR MODULATING THE VAGUS NERVE (10^{th} th CRANIAL NERVE) USING MODULATED ELECTRICAL PUSES AND AN INDUCTIVELY COUPLED STIMULATION SYSTEM”, which is a continuation of application Ser. No.10/142,298 filed on May 9, 2002. The prior applications being incorporated herein in entirety by reference, and priority is claimed from these applications.

This application is also related to application Ser. No. 10/921,757 filed Aug. 19, 2004, entitled “METHOD AND SYSTEM TO PROVIDE THERAPY FOR NEUROPSYCHIATRIC DISORDERS AND COGNITIVE IMPAIRMENTS USING GRADIENT MAGNETIC PULSES TO THE BRAIN AND PULSED ELECTRICAL STIMULATION TO VAGUS NERVE(S)”.

### FIELD OF INVENTION

This invention relates to providing magnetic and electrical pulses to the body, more specifically using combination of repetitive transcranial magnetic stimulation (rTMS) to the brain, and electrical pulses to vagus nerve(s) to provide therapy for neuropsychiatric disorders, and cognitive impairments.

### BACKGROUND

This disclosure is directed to method and system for providing adjunct (add-on) therapy for neuropsychiatric disorders and cognitive impairments, including depression, bipolar depression, anxiety disorders, obsessive-compulsive disorders, schizophrenia, borderline personality disorders, sleep disorders, learning difficulties, memory impairments and the like. The method and system comprises using combination of repetitive transcranial magnetic stimulation (rTMS) to the brain, and providing electrical pulses for stimulation and/or blocking to vagus nerve(s), to provide therapy. rTMS and VNS may be used in combination with drug therapy. An object of this invention is to provide combined/synergistic benefits of the two therapies, i.e. rTMS and VNS.

The combination use of rTMS and VNS is depicted in conjunction with FIG. 1, and may be in any order, any combination or any sequence as determined by the physician. In the method of this application, the beneficial effects of rTMS and VNS would be synergistic or at least additive. The rationale for the combined systems is that with rTMS the electromagnetic energy is penetrated from outside to inside in changing magnetic fields, and with VNS the electrical pulses are delivered to the vagus nerve(s) 54, which provides stimulation (neuromodulation) from inside (i.e. from vagus nerve to brain-stem to other projections in the brain). Electrical pulses to the vagus nerve(s) 54 are supplied using a pulse generator means and a lead with electrodes in contact with nerve tissue. The two stimulation therapies may be applied in any combination, sequence, or time interval. rTMS are typically applied in short sessions. Vagus nerve(s) stimulation is typically applied 24 hours/day, 7 days a week, in repeating cycles. The time periods of either rTMS or VNS may vary by any amount at the discretion of the physician.

Advantageously, the two types of stimulations approach the relevant centers in the brain via different approaches. Shown in conjunction with FIG. 2 the efficacy and invasiveness of the two stimulation therapies are also matched to provide the patient with balanced risk/benefit ratio. rTMS typically provides immediate benefits of mood improvement and no known side effects, but the benefits may or may not be very long lasting. With VNS the time profile of anti-depressant benefits are sustained over a long period of time, even though they may be slow to accumulate. Therefore, advantageously the combined benefits are both immediate and long lasting, providing a more ideal therapy profile, and cover a broader spectrum of patient population.

With rTMS the approach is via supplying magnetic fields leading to electrical fields from the outside, and with vagus nerve(s) 54 pulsed electrical stimulation, the approach to centers in the brain is from the inside (FIG. 4). Shown in conjunction with FIG. 3, which is an overall diagram of the brain, and in conjunction with FIGS. 4 and 5, afferent electrical neuromodulation of the vagus nerve(s) reaches the centers in the brain via projection from the Nucleus of the Solitary Tract (FIG. 5).

As mentioned previously, any combination, or sequence, or time intervals of these two energies may be applied, and is considered within the scope of the invention.

### BACKGROUND OF DEPRESSION

Depression is a very common disorder that is often chronic or recurrent in nature. It is associated with significant adverse consequences for the patient, patient's family, and society. Among the consequences of depression are functional impairment, impaired family and social relationships, increased mortality from suicide and comorbid medical disorders, and patient and societal financial burdens. Depression is the fourth leading cause of worldwide disability and is expected to become the second leading cause by 2020.

Among the currently available treatment modalities include, pharmacotherapy with antidepressant drugs (ADDs), specific forms of psychotherapy, and electroconvulsive therapy (ECT). ADDs are the usual first line treatment for depression. Commonly the initial drug selected is a selective serotonin reuptake inhibitor (SSRI) such as fluoxetine (Prozac), or another of the newer ADDs such as venlafaxine (Effexor).

Several forms of psychotherapy are used to treat depression. Among these, there is good evidence for the efficacy of cognitive behavior therapy and interpersonal therapy, but these treatments are used less often than are ADDs. Phototherapy is an additional treatment option that may be appropriate monotherapy for mild cases of depression that exhibit a marked seasonal pattern

Many patients do not respond to initial antidepressant treatment. Furthermore, many treatments used for patients who do not respond at all, or only respond partially to the first or second attempt at antidepressant therapy are poorly tolerated and/or are associated with significant toxicity. For example, tricyclic antidepressant drugs often cause anticholinergic effects and weight gain leading to premature discontinuation of therapy, and they can by lethal in overdose (a significant problem in depressed patients). Lithium is the augmentation strategy with the best published evidence of efficacy (although there are few published studies documenting long-term effectiveness), but lithium has a narrow therapeutic index that makes it difficult to administer; among the risks associated with lithium are renal and thyroid toxicity. Monoamine oxidase inhibitors are prone to produce an interaction with certain common foods that results in hypertensive crises. Even selective serotonin reuptake inhibitors can rarely produce fatal reaction in the form of a serotonin syndrome.

Physicians usually reserve electroconvulsive therapy (ECT) for treatment-resistant cases or when they determine a rapid response to treatment is desirable. ECT is also associated with significant risks: long-lasting cognitive impairment following ECT significantly limits the acceptability of ECT as a long-term treatment for depression. Therefore, there is a compelling unmet need for non-pharmacological well-tolerated and effective long-term or maintenance treatments for patients who do not respond fully, or for patients who do not sustain a response to first-line pharmacological therapies.

FIG. 2 (shown in table form) generally highlights some of the advantages and disadvantages of various forms of nonpharmalogical interventions for the treatment of depression. For example, deep brain stimulation is regionally very specific which is good, but on the other hand requires very invasive surgical procedure. As another example, ECT has clinical applicability in the short run, but on the other hand is associated with long-lasting cognitive impairments. Considering the advantages and disadvantages of different existing treatments, as shown in conjunction with FIG. 2, a combination of rTMS therapy which involves changing magnetic fields and pulsed electrical vagus nerve stimulation is an ideal combination for device based interventions. Furthermore, in this unique combination, rTMS induces stimulation from outside, and vagus nerve stimulation (VNS) approaches the stimulation from inside the brain, as shown in conjunction with FIG. 1. The initiation and delivery of these two interventions may be in any sequence or combination, and may be in addition to any drug therapy. For example, a patient implanted with vagal nerve stimulator may be given rTMS therapy, or alternatively a patient receiving rTMS therapy may be implanted with a vagus nerve stimulator. Of course, this may be in addition to any drug therapy that may be given to a patient.

In some patients the beneficial effects of rTMS may last for sometime. These patient's may be implanted with the nerve stimulator sometime after receiving their last dose of rTMS therapy. Typically patients who have received TMS, and need a more aggressive therapy for treatment would be provided VNS. This form of combination therapy, where a patient receives rTMS therapy initially and sometime later receives pulsed electrical stimulation therapy, is also intended to be covered in the scope of the invention.

Based on this type of thinking as shown in conjunction with Table 2 below, which highlights Transcranial Magnetic Stimulation (TMS) and vagus nerve stimulation provides an ideal combination of nonpharmalogical interventions. This combination balances the invasiveness, regional specificity and clinical applicapbility, and may be with or without concomitant drug therapy.

TABLE 2
Nonpharmacological interventions for the treatment of Depression

| | Regionally | Clinically | |
|---|---|---|---|
| Intervention | specific | applicable | Invasive |
| Transcranial magnetic | ++++ | +++ | + (painful at high |
| stimulation | | | intensities) |
| Vagus nerve | ++ | +++ | +++ (surgery for |
| stimulation | | | generator implant) |

Depression is thought to involve dysregulation in a collection of brain structures, some of which are deep and not directly accessible to the TMS coil, and advantageously vagus nerve stimulation/modulation approaches the stimulation from inside the brain, as shown in conjunction with FIGS. 4 and 5. In the method of this invention, it is the synergistic/additive effects of rTMS and VNS interventions that deliver the therapy.

### PRIOR ART

Prior art is generally directed either to transcranial magnetic stimulation or to vagus nerve stimulation.

U.S. patent application 2003/0028072 (Fischell et al.) is generally directed to low frequency magnetic neurostimulator for the treatment of neurological disorders. In this disclosure an implantable embodiment applies direct electrical stimulation to electrodes implanted in or on the patient's brain, while a non-invasive embodiment causes a magnetic field to induce electrical currents in the patient's brain. There is no disclosure or suggestion for synergistic use of transcranial magnetic stimulation and vagus nerve electrical stimulation.

U.S. Pat. No. 6,132,361 (Epstein et al.) and U.S. Pat. No. 6,425,852 ( Epstein et al.)are generally directed to an improved apparatus for transcranial magnetic stimulation. The apparatus of '852 disclosure allows an improved method for active localization of language function, and can also be used in rapid rate transcranial magnetic stimulation (TMS) for the treatment of depression. There is no disclosure or suggestion for combining TMS and pulsed electrical stimulation to vagus nerve(s) for providing therapy for neuropsychiatric disorders.

U.S. Pat. No. 6,827,681 B2 (Tanner et al.) is generally directed to method and a device for transcranial stimulation and for localizing specific areas of the brain. There is no disclosure or suggestion for combining TMS and pulsed electrical stimulation to vagus nerve(s) for providing therapy for neuropsychiatric disorders.

Other prior art such as U.S. Pat. No. 6,849,040 B2 (Ruohonen et al.) and U.S. Pat. No. 5,769,778 (Abrams et al.) are generally directed to transcranial magnetic stimulation, but there is no disclosure or suggestion for combining TMS and pulsed electrical stimulation to vagus nerve(s) for providing therapy for neuropsychiatric disorders.

U.S. Pat. No. 5,299,569 (Wernicke et al.) is directed to the use of implantable pulse generator technology for treating and controlling neuropsychiatric disorders including schizophrenia, depression, and borderline personality disorder.

U.S. Pat. No. 6,205,359 B1 (Boveja) and U.S. Pat. No. 6,356,788 B2 (Boveja) are directed to adjunct therapy for neurological and neuropsychiatric disorders using an implanted lead-receiver and an external stimulator.

### SUMMARY OF THE INVENTION

This invention is directed to providing therapy or alleviating the symptoms of neuropsychiatric disorders and cognitive impairments by, providing repetitive transcranial magnetic stimulation (rTMS) to the brain and afferent neuromodulation of the vagus nerve(s) with electrical pulses. The combination of rTMS and vagus nerve stimulation (VNS) provides a more ideal combination for device based interventions, with or without concomitant drug therapy. In this novel method of therapy, rTMS induces stimulation from the outside, and selective vagus nerve stimulation approaches the stimulation from inside the brain.

Accordingly in one aspect of the invention, method and system to provide therapy for or alleviate the symptoms of neuropsychiatric disorders and cognitive impairments comprises providing rTMS to the brain of a patient and afferent neuromodulation of a vagus nerve(s) with electrical pulses.

In another aspect of the invention, the combination of rTMS provided to the brain and electrical pulses provided to vagus nerve(s) are in any sequence or any combination, as determined by the physician.

In another aspect of the invention, rTMS pulses have a frequency of about 1 Hz, 5 Hz, 20 Hz, or 60 Hz.

In another aspect of the invention, vagus nerve pulsed electrical stimulation is provided to patients that have received rTMS in the past.

In another aspect of the invention, vagus nerve pulsed electrical stimulation is provided to patients who are currently receiving rTMS, and/or drug therapy.

In another aspect of the invention, the TMS generators induce peak voltages and currents that are on the order of 2,000V and 10,000 A, respectively.

In another aspect of the invention, the afferent modulation of the vagus nerve(s) is by providing electric pulses at any point along the length said vagus nerve(s).

In another aspect of the invention, the vagus nerve(s) is/are modulated unilaterally or bilaterally.

In another aspect of the invention, the system to provide electrical pulses to the vagus nerve(s) has both implanted and external components, and may be one selected from the following group: a) an implanted stimulus-receiver with an external stimulator; b) an implanted stimulus-receiver comprising a high value capacitor for storing charge, used in conjunction with an external stimulator; c) a programmer-less implantable pulse generator (IPG) which is operable with a magnet; d) a microstimulator; e) a programmable implantable pulse generator (IPG); f) a combination implantable device comprising both a stimulus-receiver and a programmable IPG; and g) an IPG comprising a rechargeable battery.

In yet another aspect of the invention, the system for providing electrical pulses to the vagus nerve(s)can be remotely interrogated or remotely programmed over a wide area network, either wirelessly or over land-lines.

Various other features, objects and advantages of the invention will be made apparent from the following description taken together with the drawings.

### BRIEF DESCRIPTION OF THE DRAWINGS

For the purpose of illustrating the invention, there are shown in accompanying drawing forms which are presently preferred, it being understood that the invention is not intended to be limited to the precise arrangement and instrumentalities shown.

FIG. 1 is a diagram depicting the concept of the invention, where a patient receives repetitive Transcranial Magnetic Stimulation (rTMS) to the brain, and pulsed electrical stimulation to vagus nerve(s) with an implanted stimulator.

FIG. 2 depicts in table form, the peculiarities of different forms of device based therapies for neuropsychiatric disorders

FIG. 3 is a diagram showing the overall structure of the brain.

FIG. 4 is a schematic diagram of the brain showing relationship of vagus nerve and solitary tract nucleus to other centers of the brain.

FIG. 5 is a simplified block diagram illustrating the connections of solitary tract nucleus to other centers of the brain.

FIG. 6 is a diagram depicting rTMS and its effect in the brain in terms of microscopic and macroscopic response

FIG. 7 depicts overall electrical circuitry and plot of current changes with rTMS.

FIG. 8A depicts geometry for field calculation.

FIG. 8B depicts a map of the electric field E induced in an unbounded conductor in a plane below a circular coil.

FIG. 9A is a diagram of simplified circuit of typical TMS stimulator and coil.

FIG. 9B depicts the TMS coil current, I, the magnetic field B.

FIG. 10 is a simplified block diagram of the major components of a TMS generator.

FIG. 11 depicts monophasic, biphasic, and polyphasic waveform of magnetic field strength.

FIG. 12 is a simplified circuit diagram of a prior art TMS generator.

FIG. 13A depicts magnetic field of a circular coil.

FIG. 13B depicts magnetic field of figure-eight coils.

FIG. 13C depicts coil windings producing a magnetic field when current is passed through it.

FIG. 14 depicts the magnetic fields penetrating through the cranium and into the brain structures.

FIG. 15 is a table highlighting clinical studies of rTMS therapy.

FIG. 16 depicts a section of the brain with areas of brain (left labels) that are linked in a highly preliminary way with some of the emotional and affiliative functions (right labels) they modulate.

FIG. 17A shows the pulse train to be transmitted to the vagus nerve.

FIG. 17B shows the ramp-up and ramp-down characteristic of the pulse train.

FIG.18 is a simplified block diagram depicting supplying amplitude and pulse width modulated electromagnetic pulses to an implanted coil.

FIG. 19 depicts a customized garment for placing an external coil to be in close proximity to an implanted coil.

FIG. 20 shows coupling of the external stimulator and the implanted stimulus-receiver.

FIG. 21 is a schematic diagram of the implantable lead.

FIG. 22 is a schematic diagram showing the implantable lead and one form of stimulus-receiver.

FIG. 23 is a schematic block diagram showing a system for neuromodulation of the vagus nerve, with an implanted component which is both RF coupled and contains a capacitor power source.

FIG. 24 is a simplified block diagram showing control of the implantable neurostimulator with a magnet.

FIG. 25 is a schematic diagram showing implementation of a multi-state converter.

FIGS. 26A-C depicts various forms of implantable microstimulators

FIG. 27 is a figure depicting an implanted microstimulator for providing pulses to vagus nerve.

FIG. 28 is a diagram depicting the components and assembly of a microstimulator.

FIG. 29 shows functional block diagram of the circuitry for a microstimulator.

FIG. 30 is a simplified block diagram of the implantable pulse generator.

FIG. 31 is a functional block diagram of a microprocessor-based implantable pulse generator.

FIG. 32 shows details of implanted pulse generator.

FIG. 33 is a diagram showing the two modules of the implanted pulse generator (IPG).

FIG. 34A depicts coil around the titanium case with two feedthroughs for a bipolar configuration.

FIG. 34B depicts coil around the titanium case with one feedthrough for a unipolar configuration.

FIG. 34C depicts two feedthroughs for the external coil which are common with the feedthroughs for the lead terminal.

FIG. 34D depicts one feedthrough for the external coil which is common to the feedthrough for the lead terminal.

FIG. 35 shows a block diagram of an implantable stimulator which can be used as a stimulus-receiver or an implanted pulse generator with rechargeable battery.

FIG. 36 is a block diagram highlighting battery charging circuit of the implantable stimulator of FIG. 35.

FIG. 37 is a schematic diagram highlighting stimulus-receiver portion of implanted stimulator of one embodiment.

FIG. 38A depicts bipolar version of stimulus-receiver module.

FIG. 38A depicts unipolar version of stimulus-receiver module.

FIG. 39 depicts power source select circuit.

FIG. 40A shows energy density of different types of batteries.

FIG. 40B shows discharge curves for different types of batteries.

FIG. 41 depicts externalizing recharge and telemetry coil from the titanium case.

FIGS. 42A and 42B depict recharge coil on the titanium case with a magnetic shield in-between.

FIG. 43 shows in block diagram form an implantable rechargable pulse generator.

FIG. 44 depicts in block diagram form the implanted and external components of an implanted rechargable system.

FIG. 45 depicts the alignment function of rechargable implantable pulse generator.

FIG. 46 is a block diagram of the external recharger.

FIG. 47 depicts an implantable system with tripolar lead for selective unidirectional blocking of vagus nerve(s) stimulation

FIG. 48 depicts selective efferent blocking in the large diameter A and B fibers.

FIG. 49 is a schematic diagram of the implantable lead with three electrodes.

FIG. 50 depicts remote monitoring of stimulation devices.

FIG. 51 is an overall schematic diagram of the external stimulator, showing wireless communication.

FIG. 52 is a schematic diagram showing application of Wireless Application Protocol (WAP).

FIG. 53 is a simplified block diagram of the networking interface board.

FIGS. 54A and 54B is a simplified diagram showing communication of modified PDA/phone with an external stimulator via a cellular tower/base station.

### DETAILED DESCRIPTION OF THE INVENTION

The following description is of the preferred mode presently contemplated for carrying out the invention. This description is not to be taken in a limiting sense, but is made merely for the purpose of describing the general principles of the invention. The scope of the invention should be determined with reference to the claims.

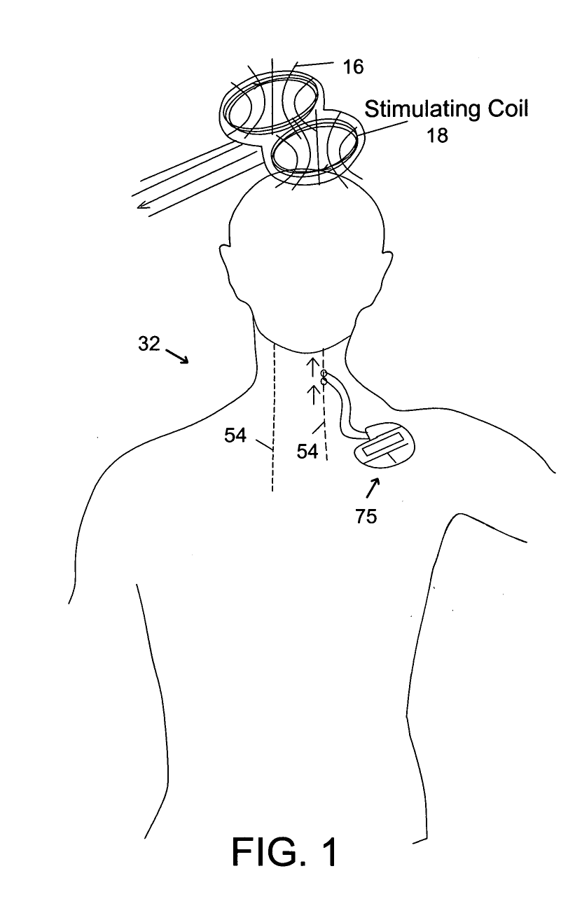

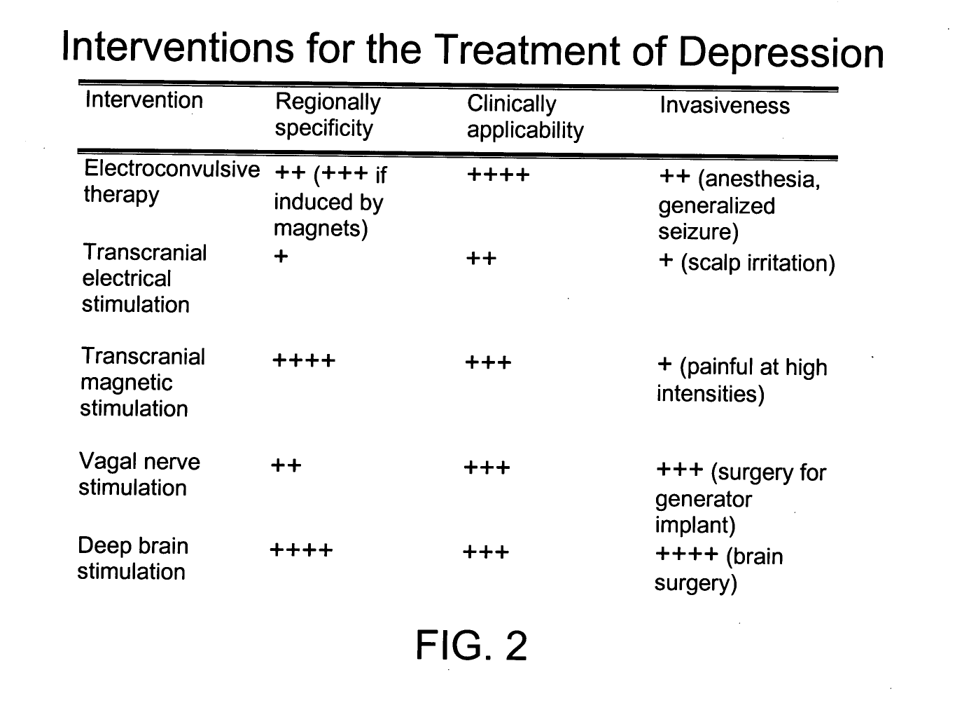

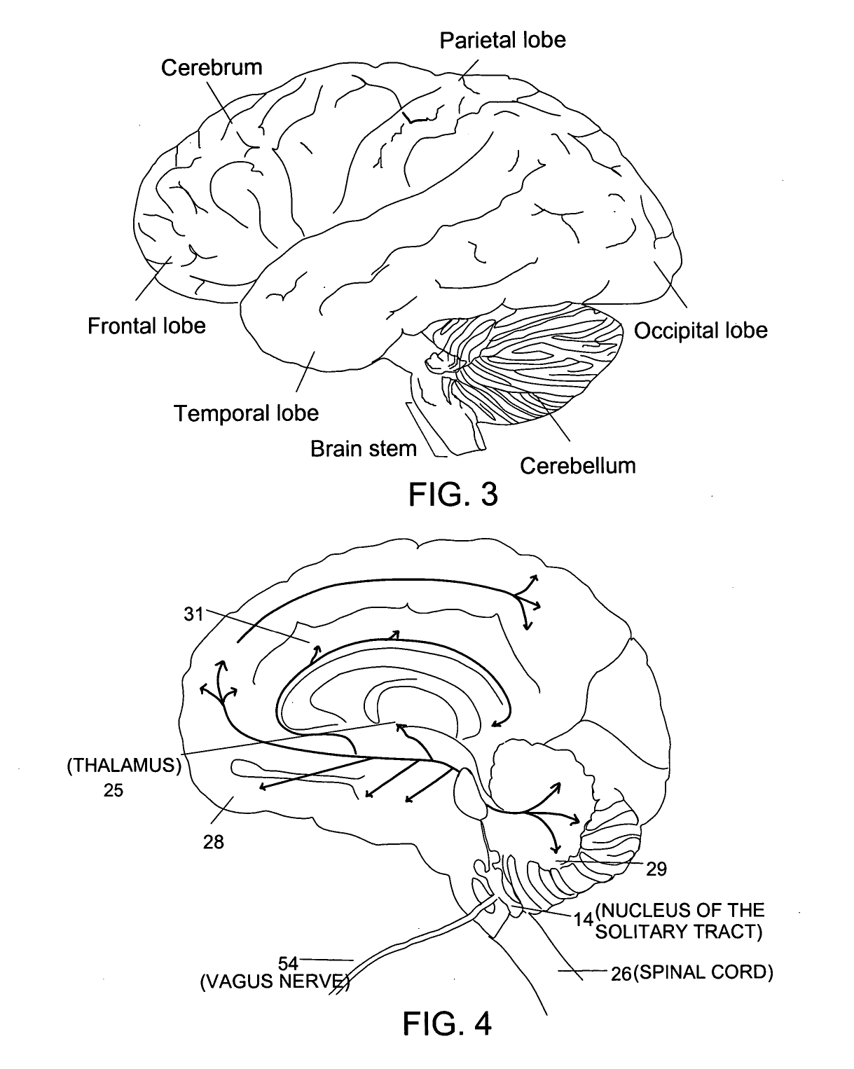

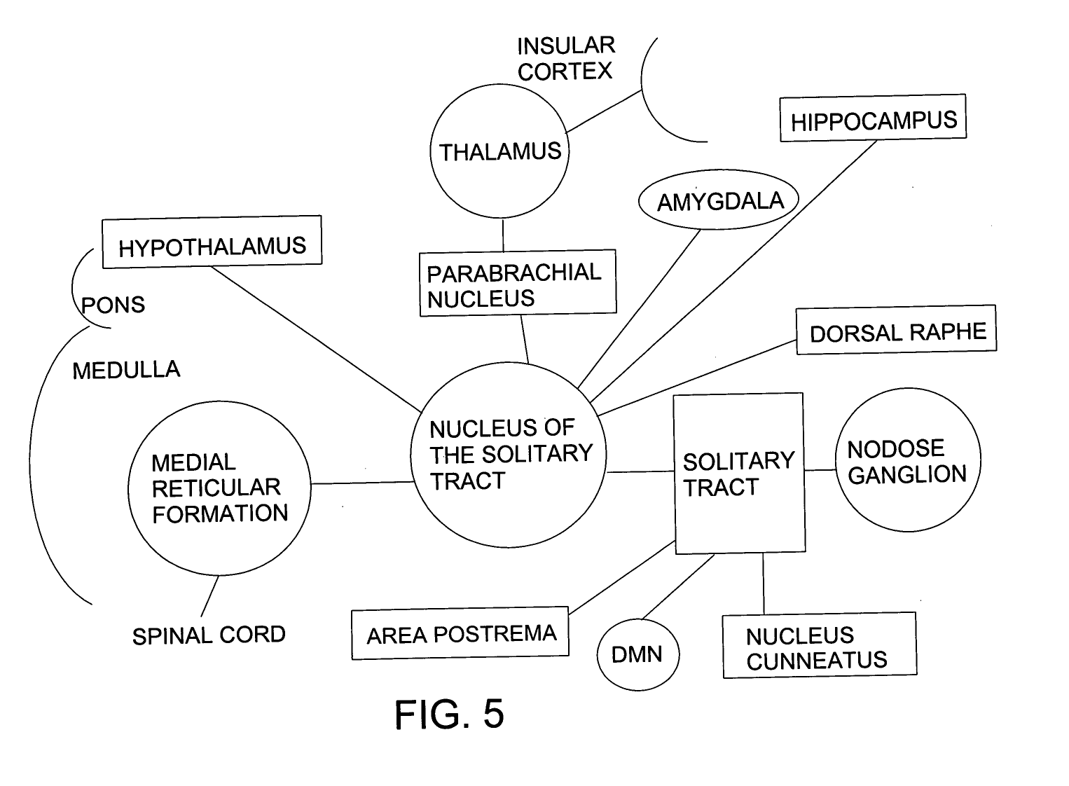

Shown in conjunction with FIG. 1 is the concept of the invention wherein repetitive transcranial magnetic stimulation (rTMS) is provided to the brain via external equipment and pulsed electrical stimulation is provided to vagus nerve(s) via implanted components such as an implanted pulse generator (IPG) and a lead with electrodes in contact with vagus nerve(s). The combination of transcranial magnetic stimulation (rTMS) and pulsed electrical stimulation to vagus nerve may be in any order, any sequence or any combination. rTMS is typically applied for a few minutes at a time. Pulsed electrical stimulation to vagus nerve(s) is typically applied around the clock in a repeating sequence, i.e. ON for a few minutes, and OFF for a few minutes.

A patient who has undergone rTMS in the past, or who is currently undergoing rTMS, may be implanted with an IPG and a lead for pulsed electrical stimulation to the vagus nerve(s), or alternatively a patient receiving vagus nerve(s) stimulation therapy may be supplemented with rTMS.

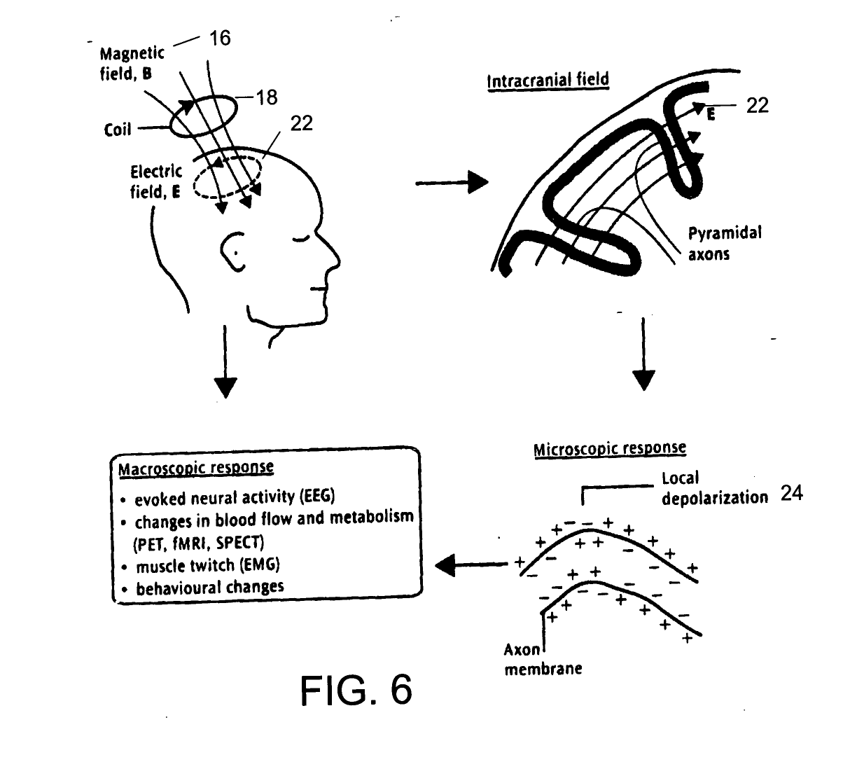

The chain of events in TMS are shown in conjunction with FIG. 6, which depicts a time-varying current in a primary circuit (coil 18) will induce an electric field 22 and thereby a current flow (eddy current) in the brain. The interaction is mediated by the magnetic field B (t) generated by the changing current in the coil. As shown in FIG. 6, the current in coil 18, generates a changing magnetic field B that induces an electric field E in the brain. The upper-right portion of the drawing depicts motor-cortex stimulation and the trajectory of the pyramidal axons. At a microscopic level, the electric field E 22 affects the transmembrane potential, which may lead to local membrane depolariztion and subsequent neural activation. As also depicted in the figure, macroscopic responses can be detected with functional imaging tools such as EEG=electroencephalography; PET=positron emission tomography; fMRI=functional magnetic resonance imaging; SPECT=single photon computed tomography with surface electromyography (EMG), or as behavioral changes.

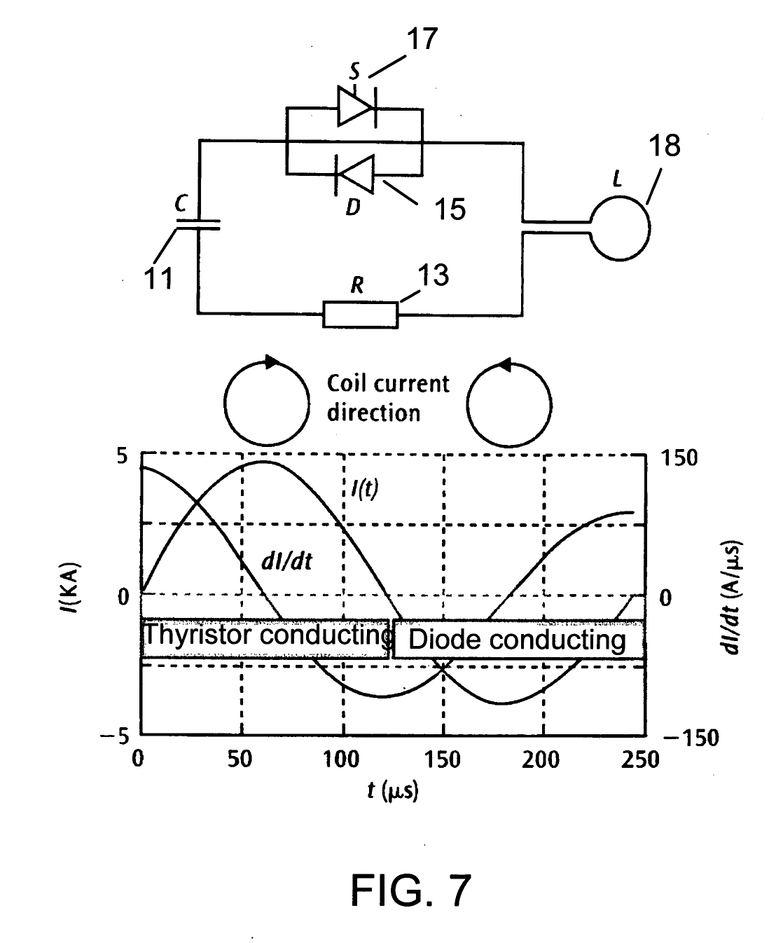

The magnetic field pulse is generated by driving a current pulse I (t) through an induction coil placed over the scalp. Shown in conjunction with FIG. 7, a basic electrical stimulator circuit consists of a capacitor 11 (capacitance C), a thyristor switch 17 and the stimulating coil 18 (inductance L). With the series resistance 13 (R) in the coil, cables, thyristor, and capacitor, the circuit forms a RCL oscillator circuit with the capacitor first charged to some Kilovolts, it is discharged through the coil 18 by gating the thyristor 17 into the conducting state. During the second half-cycle of oscillation the current in the circuit flows in the opposite direction, so that the charge returns to the capacitor 11 through the diode (D) 15. If the thyristor 17 gating is terminated during the second half-cycle, the oscillation ends when the cycle is completed.

Because of the resistive losses in the circuit, the oscillating current I (t) decays exponentially:
I(t)=(Uₒ/(Lω))e^{-αt} sin ωt

Where α=R/2L and ω²=(LC)⁻¹−α and Uₒ is the capacitor's initial voltage. The rise time of the current I (t) from zero to its peak is:
tᵣ=(1/ω)arctan(ω/α)

The electric field E and current density J=σE, σ being conductivity, induced in the tissue are proportional to dl/dt:
E(t)˜J(t)˜dl/dt=(Uₒ(Lω))e^{-αt}[ω cos ωt−α sin ωt]

The rate dl/dt jumps abruptly from zero to its peak value Uₒ/L (FIG.7). The current pulse in FIG. 7 is oscillating or biphasic; a monophasic pulse would be obtained, for example, by not including the diode or by using a large R, so that only the first half-cycle would appear.

A time-varying magnetic field B induces a primary electric field E1 according to Faraday's law:
Δ×E1=−∂B/∂t

Since the induced electric field causes a flow of current, electric charges will accumulate on any boundaries or gradients of conductivity on the path of the current. These boundary charges produce an electrostatic potential D that gives rise to a secondary electric field E₂=ΔΦ. Expressing B in terms of the vector potential A, B=Δ×A, the total electric field is
E=E₁=E₂=−∂A/∂t−ΔΦ
where Φ obeys Laplace's equation and has been solved analytically for some simple conductor shapes and numerically for more complicated shapes.

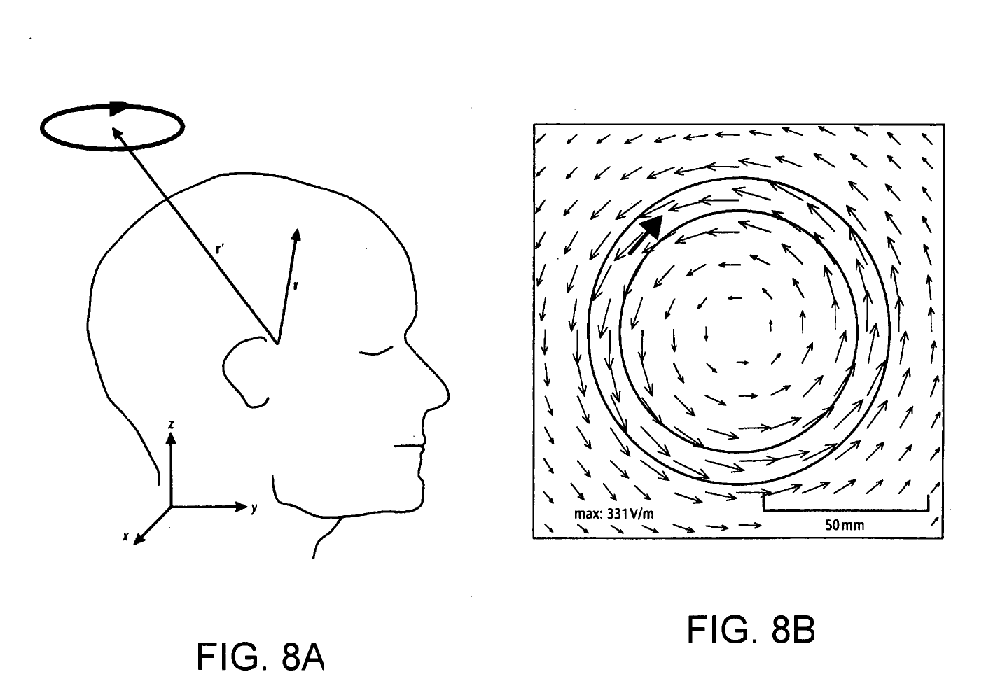

FIG. 8A depicts the geometry for field calculation. The field point inside the head is at r; the coil center is at r′. Depicted on FIG. 8B is a map of the electric field E induced in an unbounded conductor in a plane 10 mm below a circular coil wound of 11 turns. The circles depict the coil's inner and outer diameters. The thick arrow gives the direction of increasing current in the coil. Thin arrows show the direction and strength of E below the coil. The peak E is given below the figure. The rate dl/dt=100 A/μS

The electric field E sets free charges into coherent motion both in the intra-cellular and extra-cellular spaces, depolarizing or hyperpolarizing the cell membranes that interrupt the free motion of charges. In practice, the electric field strength in brain stimulation should be of the order of 100 mV/mm to elicit sufficient motor-cortex activation leading to muscle twitches. With the conductivity of the brain being about 0.4 S/m, the corresponding cortical current density would be 40 μA/mm². The understanding of the neuronal response to rTMS is very qualitative because of complex cell shapes and, for example, the effects of background neuronal activity.

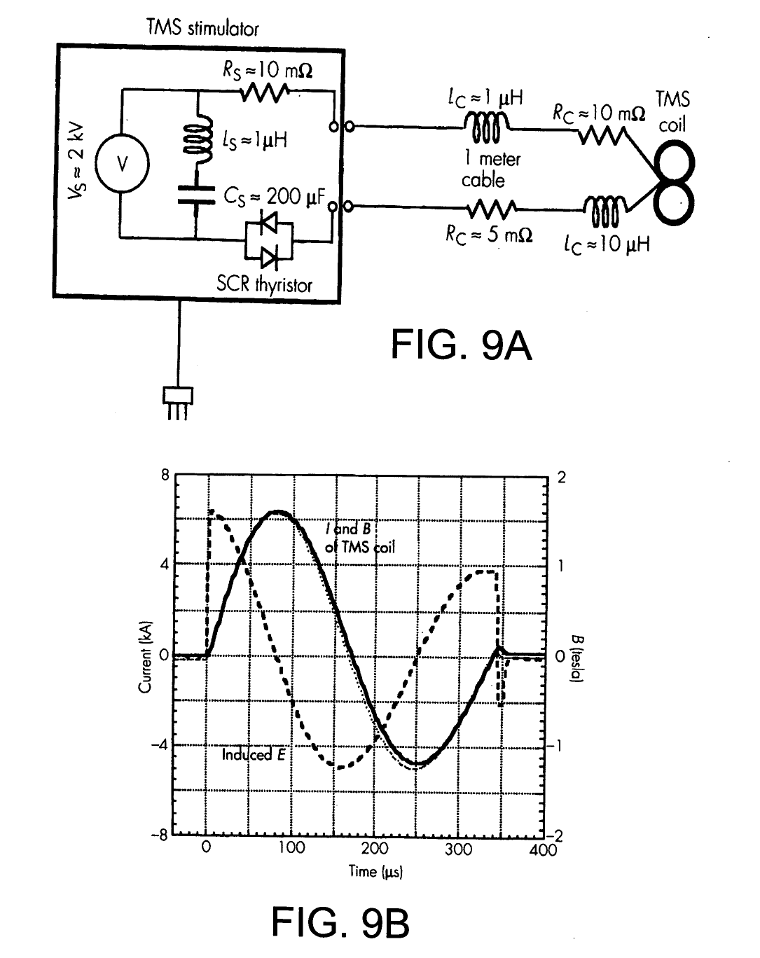

Shown in conjunction with FIG. 9A is a simplified circuit of typical TMS stimulator and coil with representative component values. The high-voltage electronic switch (thyristor) shown in FIG. 9A is crucial for creating the very short pulse needed for effective stimulation. Typical peak voltages and currents are on the order of 2,000V and 10,000 A, respectively. Another important requirement is heavy copper cable (≈1 cm in diameter) for connecting the TMS coil to the stimulator to carry the high currents involved.

FIG. 9B shows a plot of a bipolar pulse of current (I) through a TMS coil, the corresponding magnetic field (B), and the electric field (E, relative scaling) that the magnetic field induced in a small coil of wire held near it. The exact parameters of the oscillation are determined by the relative values of the storage capacitor, the inductance of the TMS coil, and the circuit resistance. Because the current has its maximum rate of change at the instant it is switched on, the induced electric field (E, FIG. 9B) is also at its maximum at that point. As the current approaches its maximum value, its rate of increase slows, and the induced electric field drops until at the current's maximum value its rate of change and the induced electric field are both zero. The current then starts to decrease, more and more rapidly, and then, as it passes through zero and reverses direction, it is decreasing at its maximum rate, creating another peak, of opposite sign, in the electric field induced. As the fall in current slows, the electric field induced begins to increase, passing through zero as the current reaches its minimum value. At the end of the cycle, as the current increases to zero, its rate of change also increases, creating another positive pulse in the induced electric field. In effect, there are two electric field pulses, the first approximately 100 μs long, and the second about 50% longer and 30% less intense.

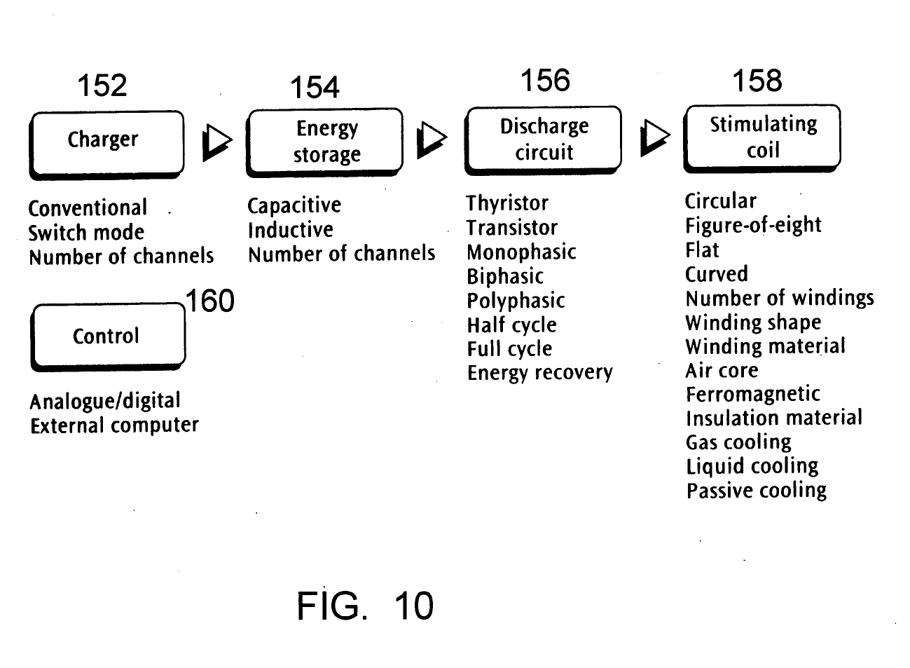

FIG. 10 shows a block diagram of the functional units of a magnetic stimulator with some of the options available in their design. The main unit utilizes a charging system 152, one or more energy storage capacitors 154, a discharge switch 156, pulse shaping, and energy recovery and control circuits 160. These parts may all be in one unit or modular and separate.

For the charging system 152, step-up transformers operating at a line frequency of 50-60 Hz may be used. Alternatively, step-up transformers operating at higher frequencies of 20 KHz or more may be used. Energy storage 154 is achieved using high-voltage capacitors. One of many capacitor types may be used. Stored energy is related to capacitance and voltage according to the following formula:
Stored energy=0.5×capacitance×(voltage)²

The important factor in the effectiveness of a magnetic nerve tissue stimulator is the maximization of the peak coil energy and a rapid magnetic field rise time. This can be achieved by using a large energy storage capacitor and/or by having an efficient energy transfer from the capacitor to the coil 158. Typically, 500 J of energy has to be transferred from the energy storage capacitor into the stimulating coil in around 100 μS or less. The impulse power output of a typical magnetic stimulator during the discharge phase can be estimated to be around 5 MW. The very high power levels require special capacitors with low internal series resistance and high peak current rating.

During the discharge, energy initially stored in the capacitor in the form of electrostatic charge is converted into magnetic energy in the stimulating coil in approximately 100 μS. This fast rate of energy transfer is necessary to achieve a rapid rate of rise of magnetic field culminating in a string pulse. To produce the necessary magnetic fields and induced currents in the tissue of the order of 10 mA/cm², the peak discharge current needs to be several thousand amps. When a magnetic stimulator receives a trigger signal, the energy stored in the energy storage capacitor is discharged into the stimulating coil using a high power switch. The stored energy, apart from that lost in the wiring and capacitor, is transferred to the coil and then returned to the instrument to reduce coil heating. In circuits with energy recovery, some or most of the energy is returned to the capacitor. The discharge switch consists of an electronic device, typically a thyristor, which is capable of switching large currents in a few microseconds. Thyristors require only a brief trigger pulse and then remain on for the duration of the current flowing in one direction. Thyristors are also used with diodes, other thyristors and passive components to shape the discharge waveform.

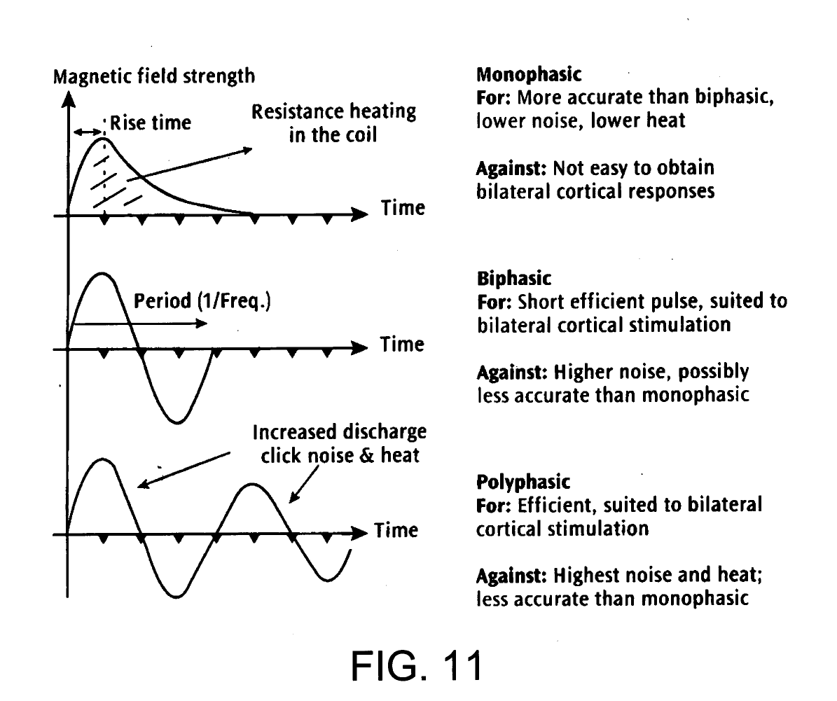

Typical magnetic field output waveforms are shown in FIG. 11. As shown in the figure monophasic, biphasic, or polyphasic waveforms may be used. For a given stored energy and magnetic field rise time, a polyphasic pulse is more effective than a biphasic pulse and a biphasic pulse is more effective than a monophasic pulse.

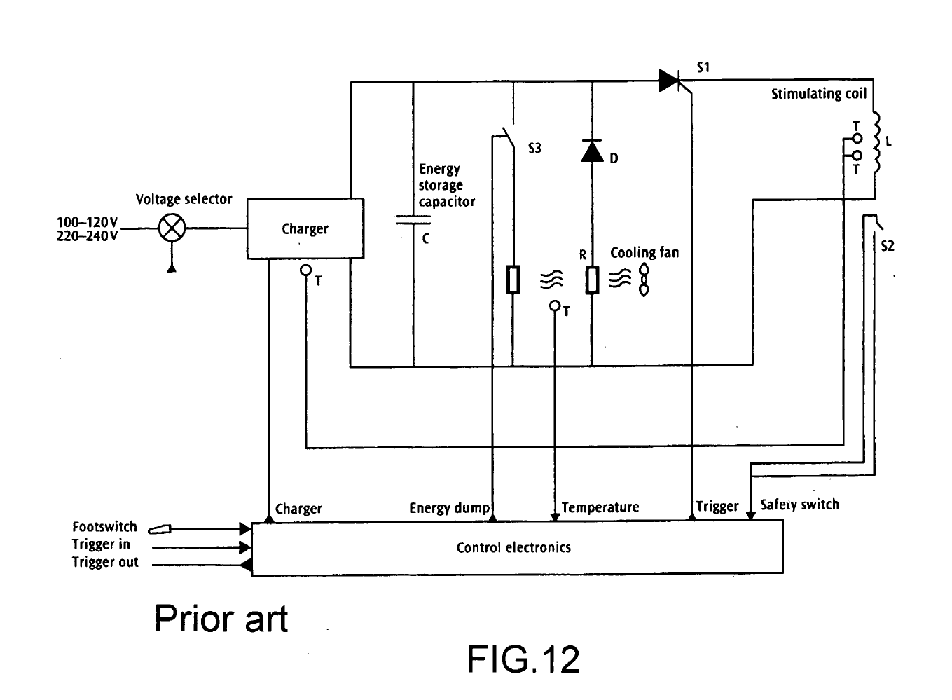

FIG. 12 shows a block diagram of a prior art monophasic magnetic stimulator. In the prior art stimulator, when thyristor Si is closed, energy stored in capacitor C is transferred to the stimulator coil L. The remaining energy is then returned to resistor R and dissipated in the form of heat. Other circuitry monitors the correct operation of the unit and dumps the stored energy in case of a fault. Other existing or to be developed TMS systems may also be used for practicing this invention. One such system that can be employed and that is well known in the art is described in U.S. Pat. No. 6,425,852, which is incorporated herein by reference in its entirety. Other TMS systems such as Cadwell (U.S.), Danteck (Denmark), Magstim (U.K.), and Schwarzer (Germany) may also be used for the purposes of this invention.

Two main coil types may used: circular coils and the figure-eight (or butterfly) coil. They are designed to achieve a peak magnetic field of 1.5-2.5 T at the face of the coil. For comparison, this is similar to the constant field in a magnetic resonance (MR) scanner, and about 40,000 times greater than the earth's magnetic field.

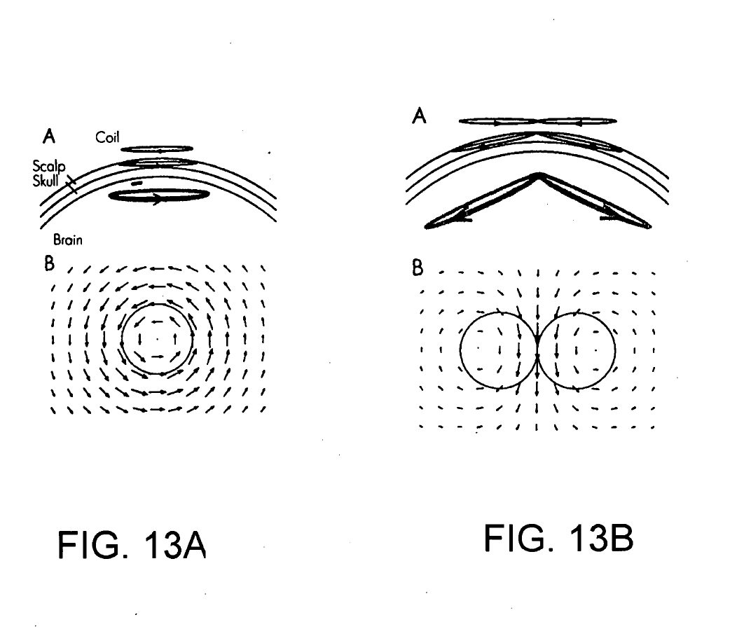

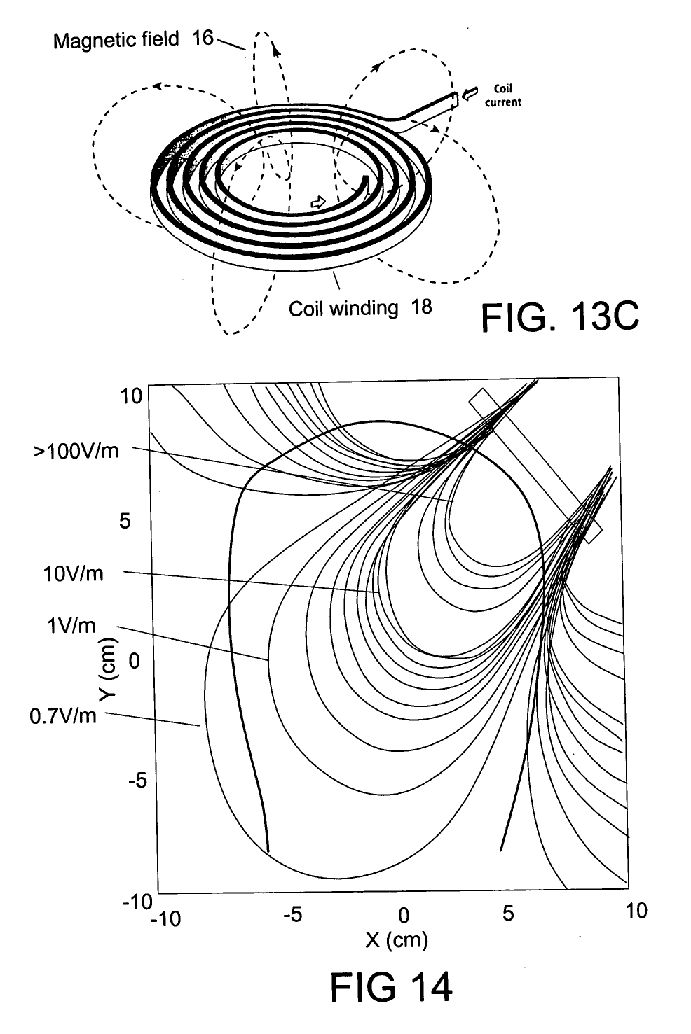

Shown in conjunction with FIG. 13A, for a circular coil, the magnetic field forms a doughnut shape around the coil, very intense near the windings and falling rapidly with distance from them. It turns out that the induced electric field in a plane below the coil is strongest in a ring the size of the coil. This is because a surface over such a circuit encloses the most magnetic flux. The flux through a small circuit near the windings would be more intense, but as it wraps around the winding, it threads back through the loop, canceling itself. A loop about the size of the coil surrounds the most flux in one direction, that is, before it starts to curve around the windings and begins canceling itself.

Circular coils are usually about 8 cm in diameter and consist of one or more turns of pure, low-resistance copper wound in a flattened doughnut configuration. In a circular coil, there is no real focus. The field is strongest adjacent to the windings and the same all around the circumference, falling rapidly with distance. The field is fairly uniform in the center of the coil but is about 30% less intense than in the area close to the windings. For a coil with radius R, the magnetic field, B, along a line perpendicular to the coil and through its center is proportional to,
B∝R²/2(R²+z²)^{3/2}

Where z is the distance from the coil along the central axis. Because the magnetic field of a simple circular coil is doughnut shaped, and its intensity rapidly decreases with distance from the loop, the sites where nerve stimulation occurs are not in the center of the loop, but at places around the loop where the patient's nerves pass close to the windings. This means that stimulation can occur at several different positions around the periphery of the coil unless it is placed on edge.

Magnetic fields can be summed, that is, the magnetic field at each point near two separate current loops is the vector sum of the magnetic field vectors from the two separate loops', hence, multiple loop configurations have been tried in attempts to improve on the penetration and focality of the field created by the circular coil. However, the superpostion of the magnetic fields of two adjacent current loops tends to make the field more uniform rather than focusing it, except where coils can be made to overlap, with currents flowing in the same direction, as in a figure-eight configuration. Because of this, only the figure-eight coil configuration has gained wide acceptance. Figure-eight coils consist of two circular or D-shaped coils mounted adjacent to each other in the same plane and wired so that their currents circulate in opposite directions. This has the effect of causing the fields of the two loops to add at their intersection, creating a cone-shaped volume of concentrated magnetic field that narrows and decreases in strength toward the apex.

For a figure-eight coil, as shown in conjunction with FIG. 13B, the magnetic flux is most intense under the intersection of the coils, forming a cone-shaped volume of concentrated magnetic flux. The two induced bands in the brain are again representative of two hollow conical figures that become wider and weaker with increasing depth but are more focal (tighter) than those of FIG. 13A. In the B portion, top view of the computed current distribution in a plane due to the same coil (two circles) and on the same scale as in A.

The stimulating coil, normally housed in molded plastic covers, consists of one or more tightly wound and well insulated copper coils together with other electronic circuitry. During the discharge of the magnetic pulse the coil winding is subjected to high voltages and currents. Although the pulse generally lasts for less than 1 ms, the forces acting on the coil winding are substantial and depend on the coil size, peak energy and construction. Careful coil design is therefore a very important aspect in the construction of a magnetic stimulator. The magnetic field produced as the current flows through a coil winding is shown in FIG. 13C. An air-cored 15-turn 90 mm coil requires approximately 8,000 A to produce a magnetic field strength of 2 Tesla at its center. Less current is required for a higher number of turns; however, coil heating is increased with small cooper wire.

Shown in conjunction with FIG. 14, is a contour plot of the fields in rTMS that are produced by a small coil some inches across, and are large and nonuniform. The rTMS magnetic field may be delivered in single-cycle sine pulses with a period of about 0.28 msec at 1-20 Hz for 20 minutes (higher frequencies may also be used). rTMS magnetic fields have strengths up to 2 T (20,000 G) at locations in the cortex falling off to less than 10 G at a distance of 20 cm away. The rTMS field consists of single-cycle cosine pulses with the same 0.28-msec period, at 1-20 Hz, similar to the magnetic field pulses. The electric field reverses sign during each pulse. The strength of the rTMS electric field ranges from more than 500 V/m in the cortex under the coil to 1 V/m 20 cm away. In the contour plot of the rTMS electric field strength (FIG. 14), it is noteworthy that the distribution of the rTMS field in the head depends greatly on the position of the coil.

Magnetic stimulation does not involve the direct passage of electric currents through the body as does electrical stimulation, but at the cellular level the mechanisms of stimulation are the same. In other words, magnetic stimulation is essentially the same as electrode-less electrical stimulation. Either directly, in the case of electrical stimulation, or indirectly, in the case of magnetic stimulation, charge is moved across an excitable cellular membrane, creating a transmembrane potential, or nerve depolarization voltage. If sufficient, this causes membrane depolarization and initiates an action potential, which then propagates along a nerve like any other action potential. The resting membrane potential of a neuron, about −70 mV (intracellular minus extracellular), is determined by the relative intra- and extracellular concentrations of sodium (Na⁺), potassium (K⁺), and chloride (Cl⁻) ions maintained by the sodium-potassium ion pump and passive diffusion. If the membrane of the neuron is depolarized from −70 mV to about 40 mV, the normally restrictive Na⁺ channels open, and the cell responds with a brief, impulsive flow of ionic current that shifts the membrane potential to +20 mV and then back to −75 mV. This response is the action potential, and the propagation of this impulse of current along the axon membrane is the mechanism by which neurons carry information.

The frequency range of TMS in the preferred embodiment may be in the range of 1-60 Hz, even though ultra-low to higher frequencies may also be used. Three frequencies of particular usefulness are 1 Hz, 10 Hz, and 20 Hz. It is known that high frequency repetitive TMS (10-20 Hz) are capable of inducing moderate to strong antidepressant effects in some individuals when administered over the left frontal cortex. Repetitive TMS using 20 Hz over the right prefrontal cortex is associated with antimanic effects, whereas the same stimulation on the left side is ineffective in mania.

Lower-frequency repetitive TMS is associated with a lateralization of antidepressant effects opposite to that found using higher frequencies, that is 1 Hz over the right prefrontal cortex appears to be associated with antidepressant effects, whereas the same parameters over the left are ineffective. Taken together, these data suggest that the relative ratio of increasing neural excitability on the left with higher frequencies and decreasing it on the right with lower frequencies may alter the ratio in favor of relative antidepressant effects, perhaps in the subgroup of patients with the classic unipolar pattern of hypofrontality.

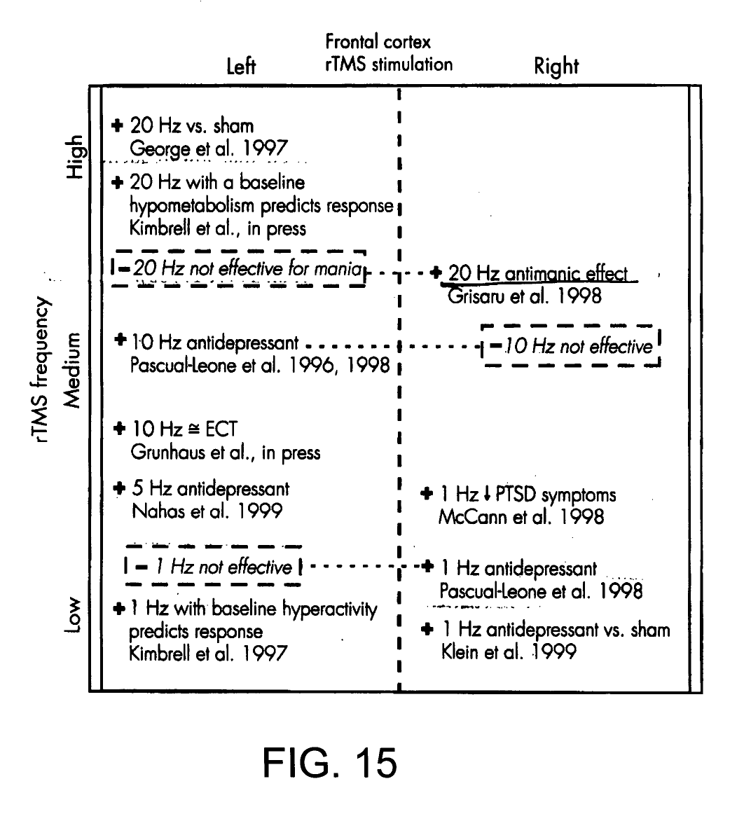

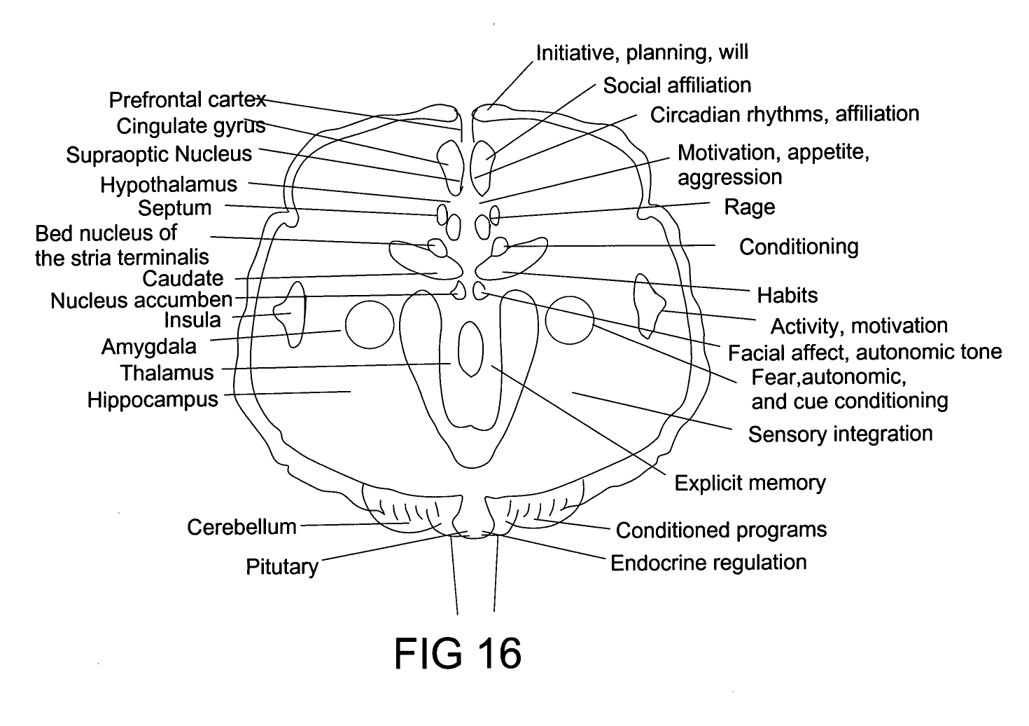

FIG. 15 summerizes numerous published clinical studies that show affective responses to repetitive TMS as a function of frequency and hemisphere laterality interaction. Clinical data shows that different frequencies of stimulation have different physiological and clinical effects, and that these different frequencies interact with laterality. In the method of this disclosure, where vagus nerve modulation is used synergistically with TMS, either low, medium or high frequencies may be used. Therefore TMS with 1 Hz, 5 Hz, 20 Hz, or 60 Hz are all considered with the scope of the invention. Shown in conjunction with FIG. 16, is a section of the brain, showing areas of brain (left labels) that are linked in a highly preliminary way with some of the emotional and affiliative functions (right labels) they modulate. The electric field shown in FIG. 14 penetrates deep in the brain to modulate activity in the deep limbic and paralimbic structures thought to modulate affective and affiliative behavior.

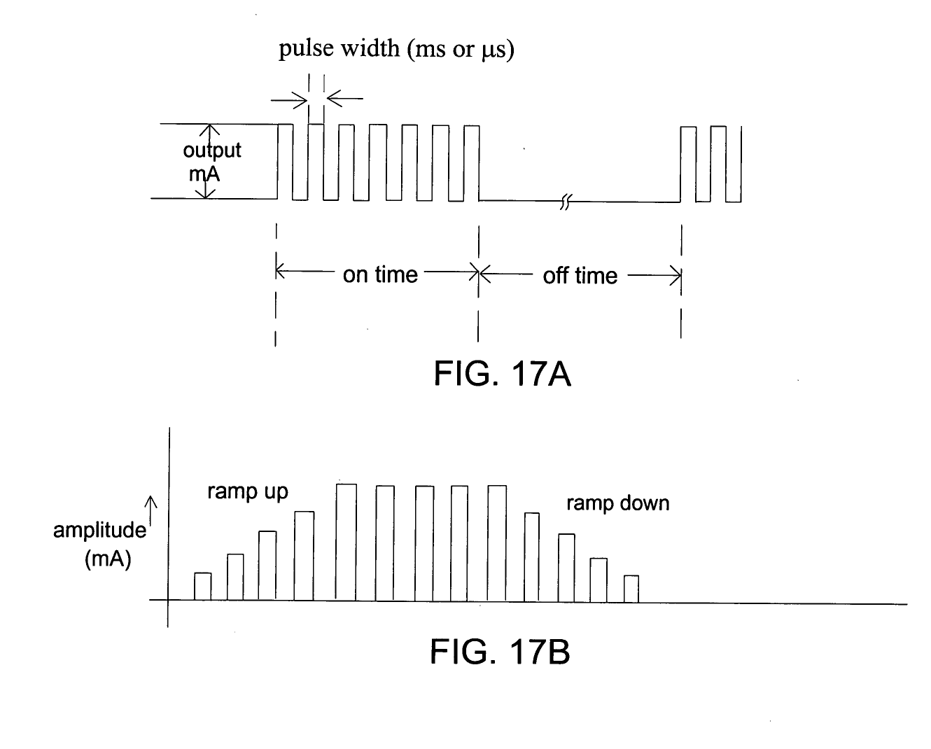

In another aspect of the invention, modulation of some autonomic centers pertinent to the psychiatric disorders, is performed by providing pulsed electrical stimulation to vagus nerve(s) 54, which is shown in FIG. 4, and which is the X^{th} cranial nerve in the body. Other cranial nerves such as trigeminal nerve, or glossopharangeal nerve could also be used for this purpose. Since vagus nerve(s) is the easiest to expose, especially at the level of the neck, it is the preferred cranial nerve. Representative pulses provided to vagus nerve(s) is shown in conjunction with FIG. 17A. Blocking pulses to selected branches may also be provided as disclosed later.

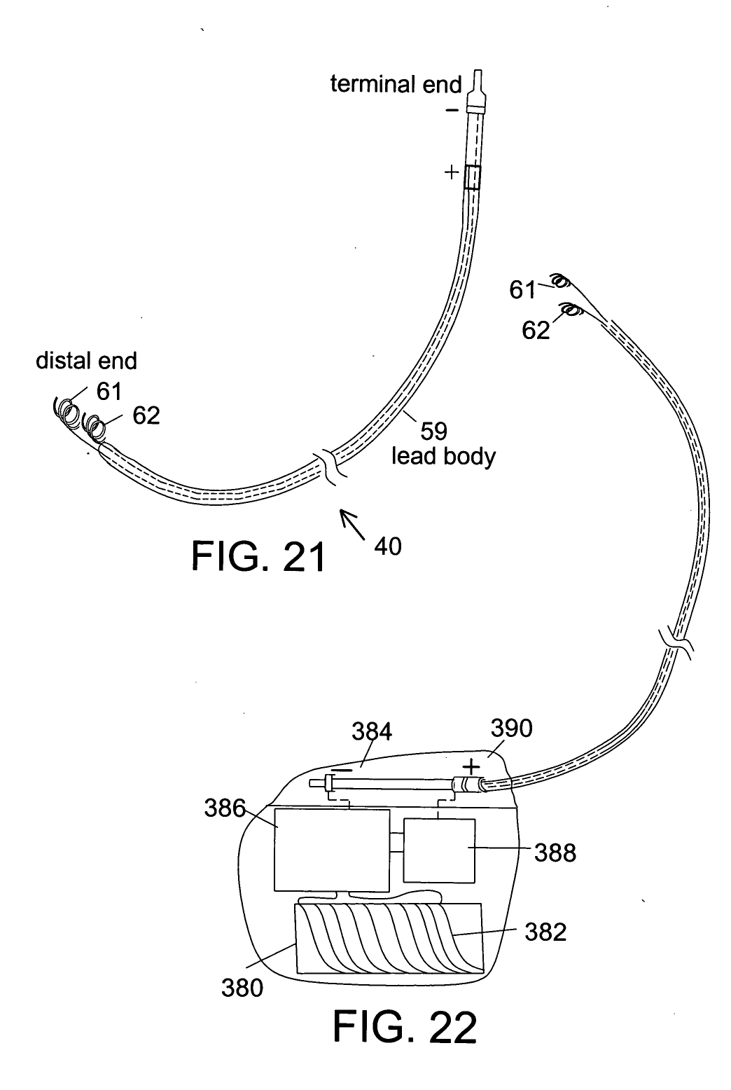

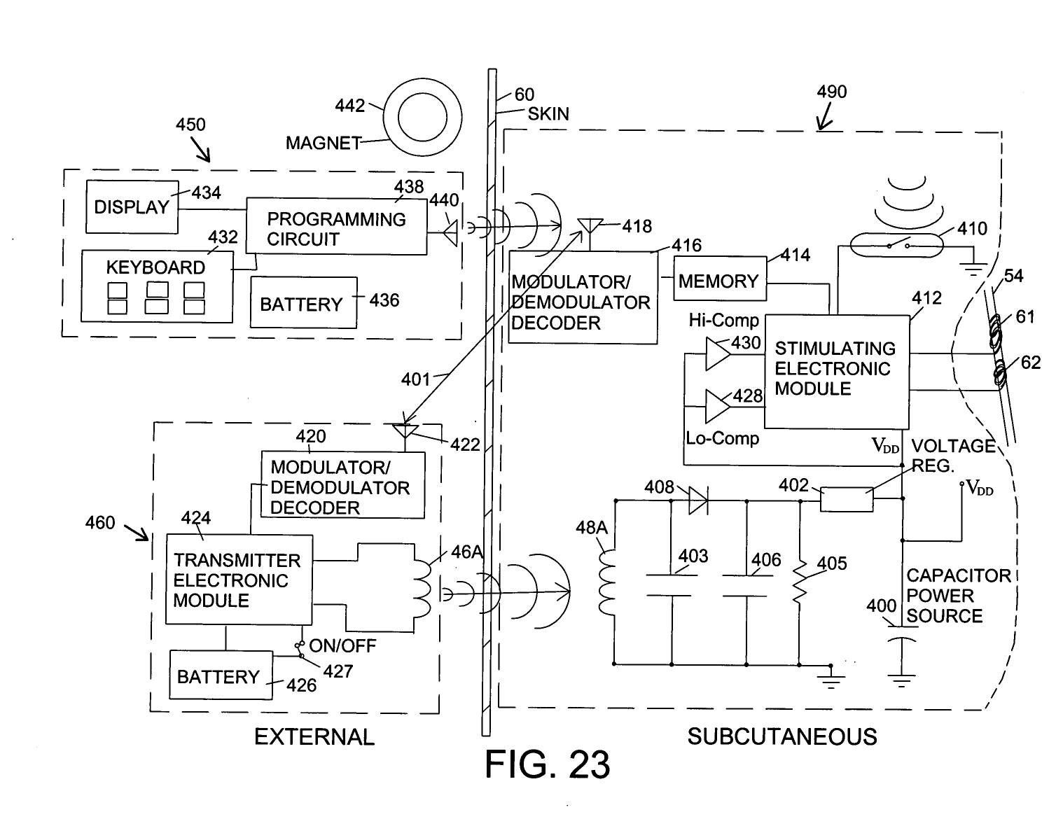

As was shown in conjunction with FIG. 1, pulsed electrical stimulation to the vagus nerve(s) 54 is provided utilizing a pulse generator means and an implanted lead 40. The implanted lead comprises a pair of electrodes 61, 62 (FIG. 21) that are adapted to be in contact with the vagus nerve(s) 54 for directly stimulating the nerve tissue. These electrodes may be placed on the vagus nerve 54 at around the neck level or around the diaphragmatic level, either just above or below the diaphragm. Also the electrodes may be implanted on one nerve for unilateral stimulation, or on both nerves for bilateral stimulation. The terminal end of the lead connects to either a pulse generator or a stimulus-receiver means.

Electrical pulses are provided to the vagus nerve(s) 54 using a system that comprises both implantable and external components. The system to provide selective stimulation (neuromodulation) may be selected from one of the following:
a) an implanted stimulus-receiver with an external stimulator;
b) an implanted stimulus-receiver comprising a high value capacitor for storing charge, used in conjunction with an external stimulator;
c) a programmer-less implantable pulse generator (IPG) which is operable with a magnet;
d) a microstimulator;
e) a programmable implantable pulse generator (IPG);
f) a combination implantable device comprising both a stimulus-receiver and a programmable IPG; and
g) an IPG comprising a rechargeable battery.

The pulse generator means is in electrical contact with a lead, which is adapted to be in contact with the vagus nerve(s) or its branches via electrodes. The pulse generator/stimulator can be of any form or type including those that are in current use, or in development, or to be developed in future. U.S. Pat. Nos. 4,702,254, 5,025,807, and 5,154,172 (Zabara) describe pulse generator and associated software to provide VNS therapy which are also included herein by reference, in this invention for application of VNS.

Using any of these systems, selective pulsed electrical stimulation is applied to vagus nerve(s) for afferent neuromodulation, at any point along the length of the nerve. The waveform of electrical pulses is shown in FIG. 17A. As shown in FIG. 17B, for patient comfort when the electrical stimulation is turned on, the electrical stimulation is ramped up and ramped down, instead of abrupt delivery of electrical pulses.

These stimulation systems for vagus nerve modulation are more fully described in a co-pending application (Ser. No. 10/841,995), but are mentioned here briefly for convenience. In each case, an implantable lead is surgically implanted in the patient 32. The vagus nerve(s) is/are surgically exposed and isolated. The electrodes on the distal end of the lead 40 are wrapped around the vagus nerve(s) 54, and the lead 40 is tunneled subcutaneously. A pulse generator means is connected to the proximal end of the lead. The power source may be external, implantable, or a combination device.

### Implanted Stimulus-Receiver with an External Stimulator

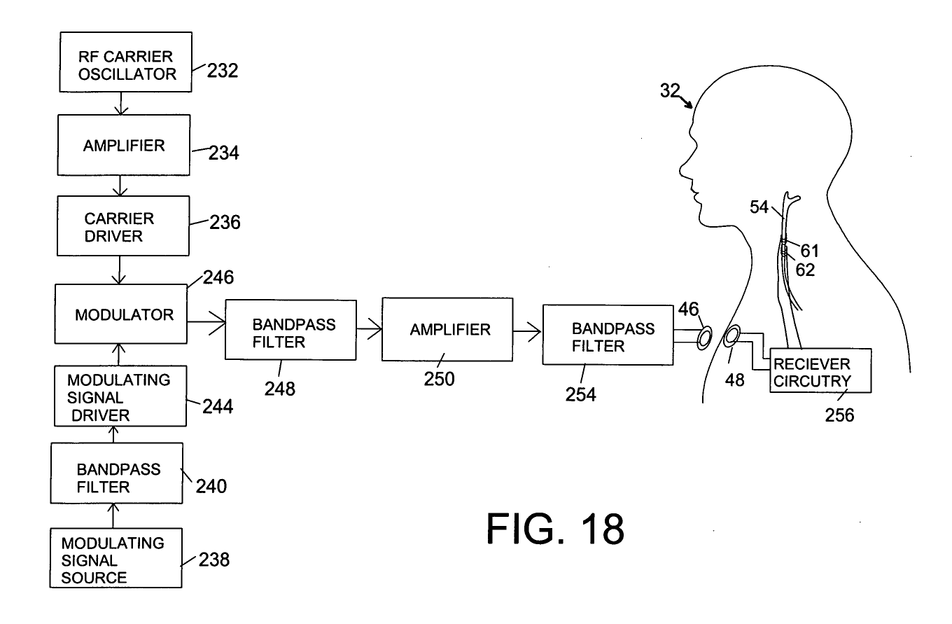

For utilizing an external power source, a passive implanted stimulus-receiver may be used. This embodiment of the vagus nerve pulse generator means is shown in conjunction with FIG. 18. A modulator 246 receives analog (sine wave) high frequency “carrier” signal and modulating signal. The modulating signal can be multilevel digital, binary, or even an analog signal. In this embodiment, mostly multilevel digital type modulating signals are used. The pulse amplitude and pulse width modulated signal is amplified 250, conditioned 254, and transmitted via a primary coil 46 which is external to the body. A secondary coil 48 of an implanted stimulus receiver, receives, demodulates, and delivers these pulses to the vagus nerve(s) 54 via electrodes 61 and 62. The receiver circuitry 256 is described later.

The carrier frequency is optimized. One preferred embodiment utilizes electrical signals of around 1 Mega-Hertz, even though other frequencies can be used. Low frequencies are generally not suitable because of energy requirements for longer wavelengths, whereas higher frequencies are absorbed by the tissues and are converted to heat, which again results in power losses.

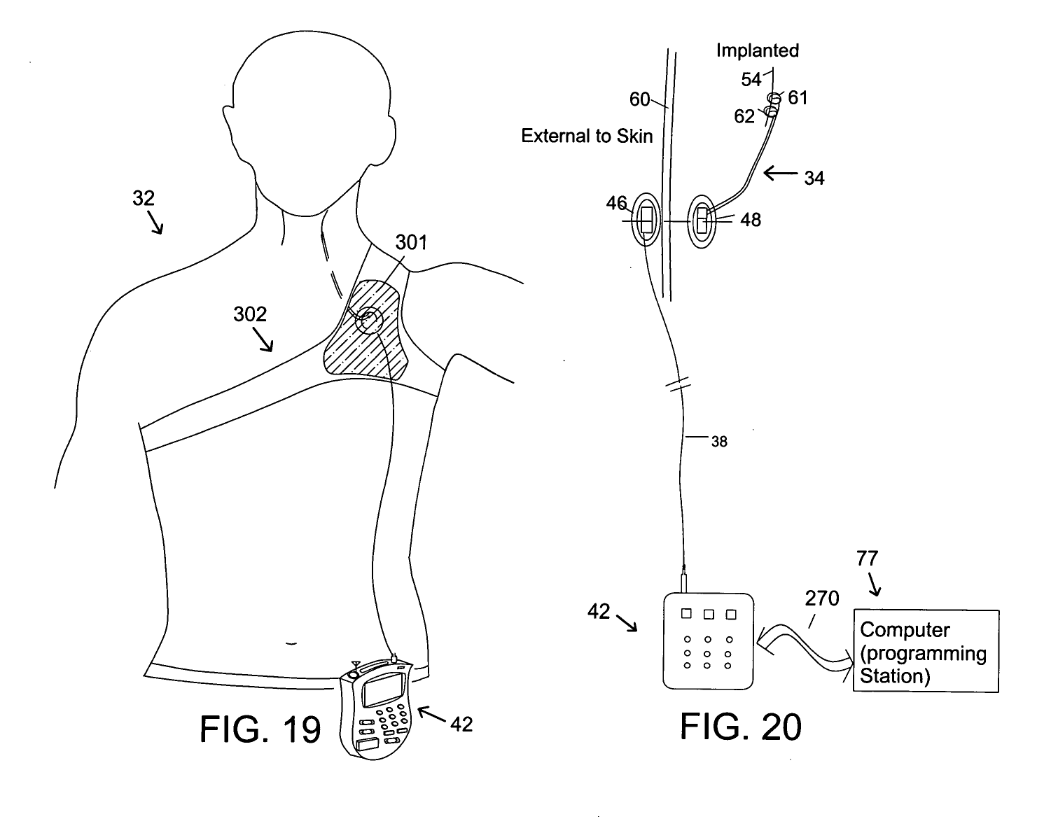

Shown in conjunction with FIG. 19, the coil for the external transmitter (primary coil 46) may be placed in the pocket 301 of a customized garment 302, for patient convenience.

Shown in conjunction with FIG. 20, the primary (external) coil 46 of the external stimulator 42 is inductively coupled to the secondary (implanted) coil 48 of the implanted stimulus-receiver 34. The implantable stimulus-receiver 34 has circuitry at the proximal end, and has two stimulating electrodes at the distal end 61,62. The negative electrode (cathode) 61 is positioned towards the brain and the positive electrode (anode) 62 is positioned away from the brain.

For therapy to commence, the primary (external) coil 46 is placed on the skin 60 on top of the surgically implanted (secondary) coil 48. An adhesive tape may be placed on the skin 60 and external coil 46 such that the external coil 46, is taped to the skin 60. For efficient energy transfer to occur, it is important that the primary (external) 46 and secondary (internal) coils 48 be positioned along the same axis and be optimally positioned relative to each other. In this embodiment, the external coil 46 may be connected to proximity sensing circuitry 50, in which case the correct positioning of the external coil 46 with respect to the internal coil 48 is indicated by turning “on” of a light emitting diode (LED) on the external stimulator 42.

The programmable parameters are stored in a programmable logic in the external stimulator 42. The predetermined programs stored in the external stimulator 42 are capable of being modified through the use of a separate programming station 77. A Programmable Array Logic Unit and interface unit are interfaced to the programming station 77. The programming station 77 can be used to load new programs, change the existing predetermined programs or the program parameters for various stimulation programs. The programming station is connected to the programmable array unit (comprising programmable array logic and interface unit) with an RS232-C serial connection. The main purpose of the serial line interface is to provide an RS232-C standard interface. Other suitable well known interface connections may also be used.

This method enables any portable computer with a serial interface to communicate and program the parameters for storing the various programs. The serial communication interface receives the serial data, buffers this data and converts it to a 16 bit parallel data. The programmable array logic component of programmable array unit (not shown) receives the parallel data bus and stores or modifies the data into a random access matrix. This array of data also contains special logic and instructions along with the actual data. These special instructions also provide an algorithm for storing, updating and retrieving the parameters from long-term memory. The programmable logic array unit, interfaces with long term memory to store the predetermined programs. All the previously modified programs can be stored here for access at any time, as well as, additional programs can be locked out for the patient. The programs consist of specific parameters and each unique program will be stored sequentially in long-term memory. A battery unit is present to provide power to all the components. The logic for the storage and decoding is stored in a random addressable storage matrix (RASM).

Conventional microprocessor and integrated circuits are used for the logic, control and timing circuits. Conventional bipolar transistors are used in radio-frequency oscillator, pulse amplitude ramp control and power amplifier. A standard voltage regulator is used in low-voltage detector. The hardware and software to deliver the pre-determined programs is well known to those skilled in the art.

The selective stimulation of the vagus nerve(s) can be performed in one of two ways. One method is to activate one of several “pre-determined/pre-packaged” programs. A second method is to “custom” program the electrical parameters, which can be selectively programmed for specific therapy to the individual patient. The electrical parameters that can be individually programmed, include variables such as pulse amplitude, pulse width, frequency of stimulation, stimulation on-time, and stimulation off-time. Table one below defines the approximate range of parameters,

TABLE 1
Electrical parameter range delivered to the nerve

| PARAMER | RANGE |
|---|---|
| Pulse Amplitude | 0.1 Volt-15 Volts |
| Pulse width | 20 μS-5 mSec. |
| Frequency | 5 Hz-200 Hz |
| On-time | 10 Secs-24 hours |
| Off-time | 10 Secs-24 hours |

The parameters in Table 1 are the electrical signals delivered to the nerve via the two electrodes 61,62 (distal and proximal) around the nerve, as shown in FIG. 20. It being understood that the signals generated by the external pulse generator 42 and transmitted via the primary coil 46 are larger, because the attenuation factor between the primary coil 46 and secondary coil 48 is approximately 10-20 times, depending upon coupling factors such as the distance, and orientation between the two coils. Accordingly, the range of transmitted signals of the external stimulator 42 may be approximately 10-20 times larger than shown in Table 1.

Referring to FIG. 21, the implanted lead component of the system is similar to cardiac pacemaker leads, except for distal portion (or electrode end) of the lead 40. The lead terminal preferably is linear bipolar, even though it can be bifurcated, and plug(s) into the cavity of the pulse generator means. The lead body 59 insulation may be constructed of medical grade silicone, silicone reinforced with polytetrafluoro-ethylene (PTFE), or polyurethane. The electrodes 61,62 for stimulating the vagus nerve 54 may either wrap around the nerve once or may be spiral shaped. These stimulating electrodes may be made of pure platinum, platinum/Iridium alloy or platinum/iridium coated with titanium nitride. The conductor connecting the terminal to the electrodes 61,62 is made of an alloy of nickel-cobalt. The implanted lead design variables are also summarized in table two below.

TABLE 2
Lead design variables

| | |
|---|---|
| Proximal | Distal |
| End | End |

| | | | Conductor | | |
|---|---|---|---|---|---|
| | | | (connecting | | |
| | Lead body- | | proximal | | |
| Lead | Insulation | | and distal | Electrode - | Electrode - |
| Terminal | Materials | Lead-Coating | ends) | Material | Type |
| Linear | Polyurethane | Antimicrobial | Alloy of | Pure | Spiral |
| bipolar | | coating | Nickel- | Platinum | electrode |
| | | | Cobalt | | |
| Bifurcated | Silicone | Anti- | | Platinum- | Wrap-around |
| | | Inflammatory | | Iridium | electrode |
| | | coating | | (Pt/lr) Alloy | |
| | Silicone with | Lubricious | | Pt/lr coated | Steroid |
| | Polytetrafluoroethylene | coating | | with Titanium | eluting |
| | (PTFE) | | | Nitride | |
| | | | | Carbon | Hydrogel |
| | | | | | electrodes |
| | | | | | Cuff |
| | | | | | electrodes |

Once the lead is fabricated, coating such as anti-microbial, anti-inflammatory, or lubricious coating may be applied to the lead body 59.

### Implanted Stimulus-Receiver Comprising a High Value Capacitor for Storing Charge, Used in Conjunction with an External Stimulator

In one embodiment, the implanted stimulus-receiver may be a system which is RF coupled combined with a power source. In this embodiment, the implanted stimulus-receiver comprises high value, small sized capacitor(s) for storing charge and delivering electric stimulation pulses for up to several hours by itself, once the capacitors are charged. The packaging is shown in FIG. 22. Using mostly hybrid components and appropriate packaging, the implanted portion of the system described below can be miniaturized. As shown in FIG. 22, a solenoid coil 382 wrapped around a ferrite core 380 is used as the secondary of an air-gap transformer for receiving power and data to the implanted device. The primary coil is external to the body. Since the coupling between the external transmitter coil and receiver coil 382 may be weak, a high-efficiency transmitter/amplifier is used in order to supply enough power to the receiver coil 382. Class-D or Class-E power amplifiers may be used for this purpose. The coil for the external transmitter (primary coil) may be placed in the pocket of a customized garment, as was shown previously in FIG. 19.

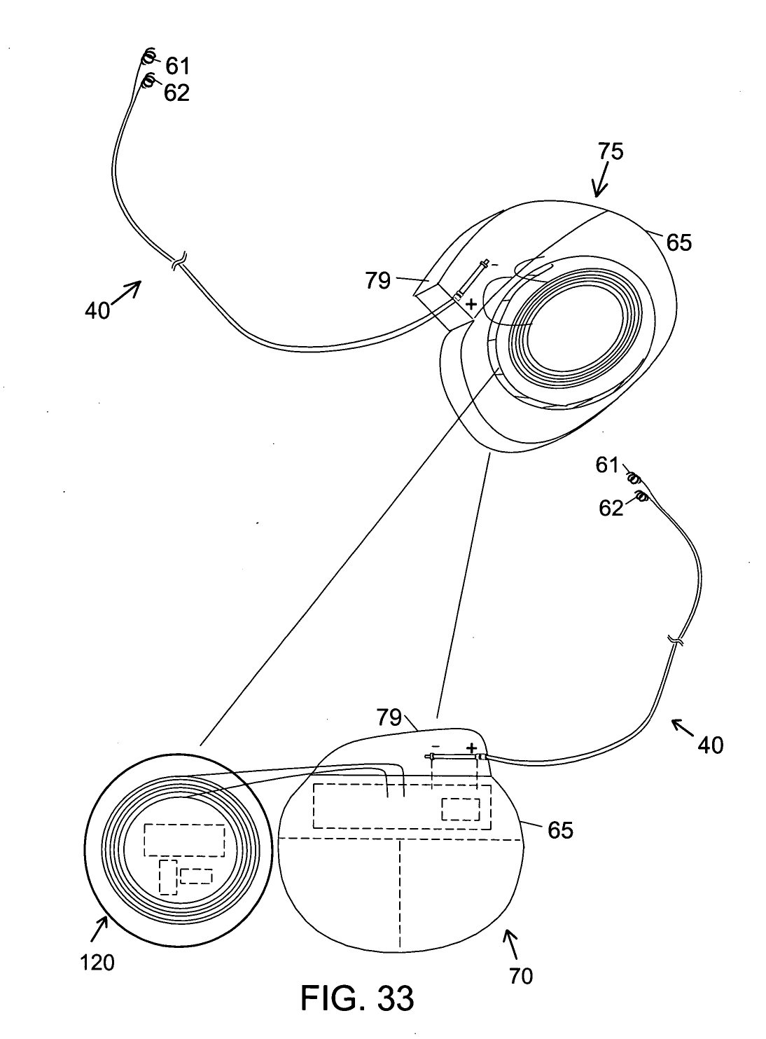

Shown in conjunction with FIG. 33 of the implanted stimulus-receiver 490 and the system, the receiving inductor 48A and tuning capacitor 403 are tuned to the frequency of the transmitter. The diode 408 rectifies the AC signals, and a small sized capacitor 406 is utilized for smoothing the input voltage V_{I} fed into the voltage regulator 402. The output voltage V_{D} of regulator 402 is applied to capacitive energy power supply and source 400 which establishes source power V_{DD}. Capacitor 400 is a big value, small sized capacative energy source which is classified as low internal impedance, low power loss and high charge rate capacitor, such as Panasonic Model No. 641.

The refresh-recharge transmitter unit 460 includes a primary battery 426, an ON/Off switch 427, a transmitter electronic module 424, an RF inductor power coil 46A, a modulator/demodulator 420 and an antenna 422.

When the ON/OFF switch is on, the primary coil 46A is placed in close proximity to skin 60 and secondary coil 48A of the implanted stimulator 490. The inductor coil 46A emits RF waves establishing EMF wave fronts which are received by secondary inductor 48A. Further, transmitter electronic module 424 sends out command signals which are converted by modulator/demodulator decoder 420 and sent via antenna 422 to antenna 418 in the implanted stimulator 490. These received command signals are demodulated by decoder 416 and replied and responded to, based on a program in memory 414 (matched against a “command table” in the memory). Memory 414 then activates the proper controls and the inductor receiver coil 48A accepts the RF coupled power from inductor 46A.

The RF coupled power, which is alternating or AC in nature, is converted by the rectifier 408 into a high DC voltage. Small value capacitor 406 operates to filter and level this high DC voltage at a certain level. Voltage regulator 402 converts the high DC voltage to a lower precise DC voltage while capacitive power source 400 refreshes and replenishes.

When the voltage in capacative source 400 reaches a predetermined level (that is V_{DD} reaches a certain predetermined high level), the high threshold comparator 430 fires and stimulating electronic module 412 sends an appropriate command signal to modulator/decoder 416. Modulator/decoder 416 then sends an appropriate “fully charged” signal indicating that capacitive power source 400 is fully charged, is received by antenna 422 in the refresh-recharge transmitter unit 460.

In one mode of operation, the patient may start or stop stimulation by waving the magnet 442 once near the implant. The magnet emits a magnetic force Lₘ which pulls reed switch 410 closed. Upon closure of reed switch 410, stimulating electronic module 412 in conjunction with memory 414 begins the delivery (or cessation as the case may be) of controlled electronic stimulation pulses to the vagus nerve(s) 54 via electrodes 61, 62. In another mode (AUTO), the stimulation is automatically delivered to the implanted lead based upon programmed ON/OFF times.

The programmer unit 450 includes keyboard 432, programming circuit 438, rechargeable battery 436, and display 434. The physician or medical technician programs programming unit 450 via keyboard 432. This program regarding the frequency, pulse width, modulation program, ON time etc. is stored in programming circuit 438. The programming unit 450 must be placed relatively close to the implanted stimulator 490 in order to transfer the commands and programming information from antenna 440 to antenna 418. Upon receipt of this programming data, modulator/demodulator and decoder 416 decodes and conditions these signals, and the digital programming information is captured by memory 414. This digital programming information is further processed by stimulating electronic module 412. In the DEMAND operating mode, after programming the implanted stimulator, the patient turns ON and OFF the implanted stimulator via hand held magnet 442 and the reed switch 410. In the automatic mode (AUTO), the implanted stimulator turns ON and OFF automatically according to the programmed values for the ON and OFF times.

Other simplified versions of such a system may also be used. For example, a system such as this, where a separate programmer is eliminated, and simplified programming is performed with a magnet and reed switch, can also be used.

### Programmer-Less Implantable Pulse Generator (IPG)

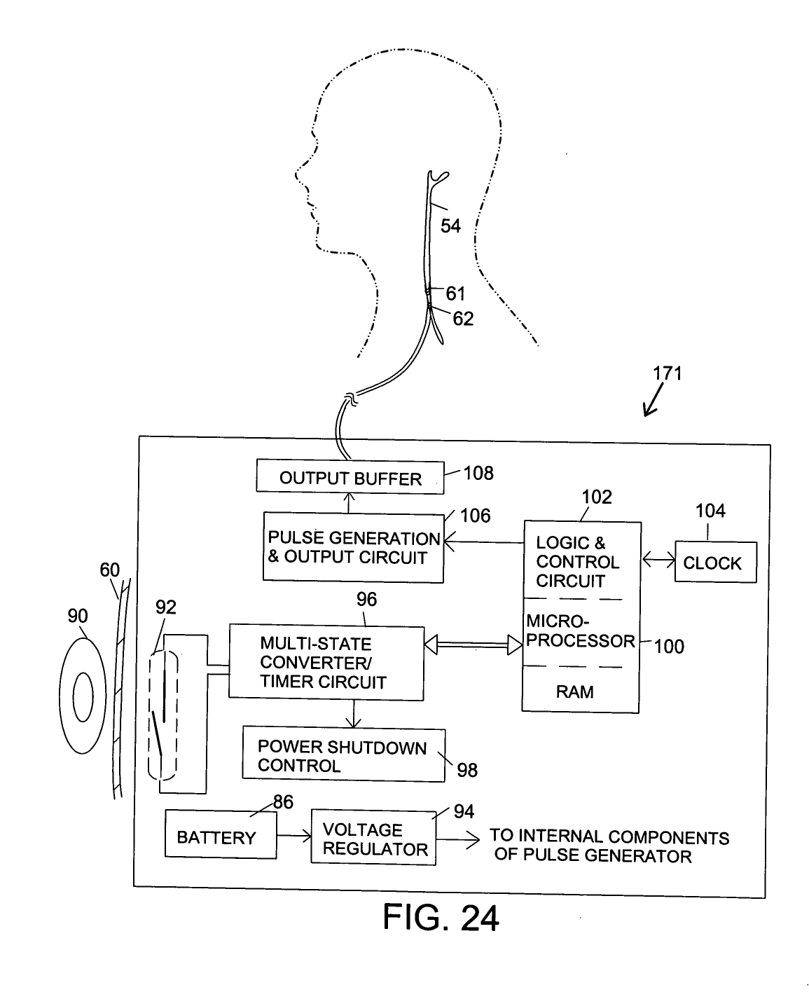

In one embodiment, a programmer-less implantable pulse generator (IPG) may be used. In this embodiment, shown in conjunction with FIG. 24, the implantable pulse generator 171 is provided with a reed switch 92 and memory & control circuitry 102. The reed switch 92 being remotely actuable by means of a magnet 90 brought into proximity of the pulse generator 171, in accordance with common practice in the art. In this embodiment, the reed switch 92 is coupled to a multi-state converter/timer circuit 96, such that a single short closure of the reed switch can be used as a means for non-invasive encoding and programming of the pulse generator 171 parameters.

In one embodiment, shown in conjunction with FIG. 25, the closing of the reed switch 92 triggers a counter. The magnet 90 and timer are ANDed together. The system is configured such that during the time that the magnet 82 is held over the pulse generator 171, the output level goes from LOW stimulation state to the next higher stimulation state every 5 seconds. Once the magnet 82 is removed, regardless of the state of stimulation, an application of the magnet, without holding it over the pulse generator 171, triggers the OFF state, which also resets the counter.

Once the prepackaged/predetermined logic state is activated by the logic and control circuit 102, the pulse generation and amplification circuit 106 deliver the appropriate electrical pulses to the vagus nerve(s) 54 of the patient via an output buffer 108 (as shown in FIG. 24). The delivery of output pulses is configured such that the distal electrode 61 (electrode closer to the brain) is the cathode, and the proximal electrode 62 is the anode. Timing signals for the logic and control circuit 102 of the pulse generator 171 are provided by a crystal oscillator 104. The battery 86 of the pulse generator 171 has terminals connected to the input of a voltage regulator 94. The regulator 94 smoothes the battery output and supplies power to the internal components of the pulse generator 171. A microprocessor 100 controls the program parameters of the device, such as the voltage, pulse width, frequency of pulses, on-time and off-time. The microprocessor 100 may be a commercially available, general purpose microprocessor or microcontroller, or may be a custom integrated circuit device augmented by standard RAM/ROM components.

In one embodiment, there are four stimulation states. A larger (or lower) number of states can be achieved using the same methodology, and such is considered within the scope of the invention. These four states are, LOW stimulation state, LOW-MED stimulation state, MED stimulation state, and HIGH stimulation state. Examples of stimulation parameters (delivered to the vagus nerve) for each state are as follows,

LOW stimulation state example is,

| | |
|---|---|
| Current output: | 0.75 milliAmps. |
| Pulse width: | 0.20 msec. |
| Pulse frequency: | 20 Hz |
| Cycles: | 20 sec. on-time and 2.0 min. off-time |
| | in repeating cycles. |

LOW-MED stimulation state example is,

| | |
|---|---|
| Current output: | 1.5 milliAmps, |
| Pulse width: | 0.30 msec. |
| Pulse frequency: | 25 Hz |
| Cycles: | 1.5 min. on-time and 20.0 min. off-time |
| | in repeating cycles. |

MED stimulation state example is,

| | |
|---|---|
| Current output: | 2.0 milliAmps. |
| Pulse width: | 0.30 msec. |
| Pulse frequency: | 30 Hz |
| Cycles: | 1.5 min. on-time and 20.0 min. off-time |
| | in repeating cycles. |

HIGH stimulation state example is,

| | |
|---|---|
| Current output: | 3.0 milliAmps, |
| Pulse width: | 0.40 msec. |
| Pulse frequency: | 30 Hz |
| Cycles: | 2.0 min. on-time and 20.0 min. off-time |
| | in repeating cycles. |

These prepackaged/predetermined programs are mearly examples, and the actual stimulation parameters will deviate from these depending on the patient or treatment application.

It will be readily apparent to one skilled in the art, that other schemes can be used for the same purpose. For example, instead of placing the magnet 90 on the pulse generator 171 for a prolonged period of time, different stimulation states can be encoded by the sequence of magnet applications. Accordingly, in an alternative embodiment there can be three logic states, OFF, LOW stimulation (LS) state, and HIGH stimulation (HS) state. Each logic state again corresponds to a prepackaged/predetermined program such as presented above. In such an embodiment, the system could be configured such that one application of the magnet 90 triggers the generator into LS State. If the generator is already in the LS state then one application triggers the device into OFF State. Two successive magnet applications triggers the generator into MED stimulation state, and three successive magnet applications triggers the pulse generator in the HIGH Stimulation State. Subsequently, one application of the magnet while the device is in any stimulation state, turns the device OFF.

The advantage of this embodiment is that it is cheaper to manufacture than a fully programmable implantable pulse generator (IPG).

### Microstimulator

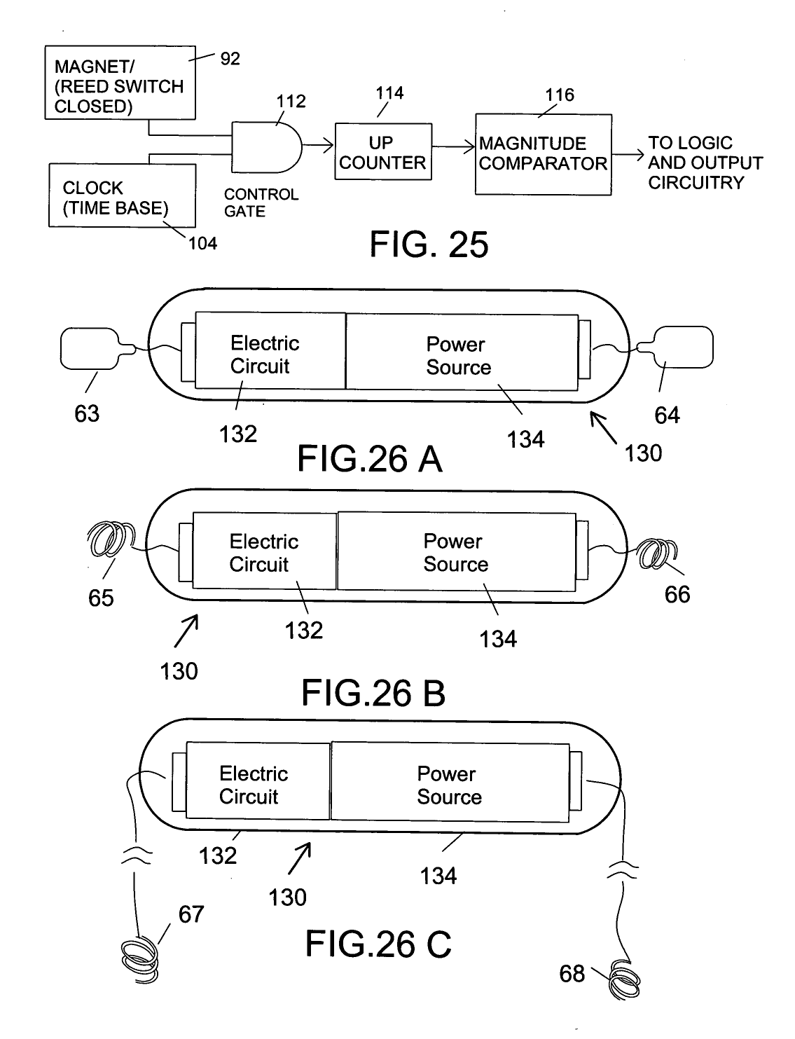

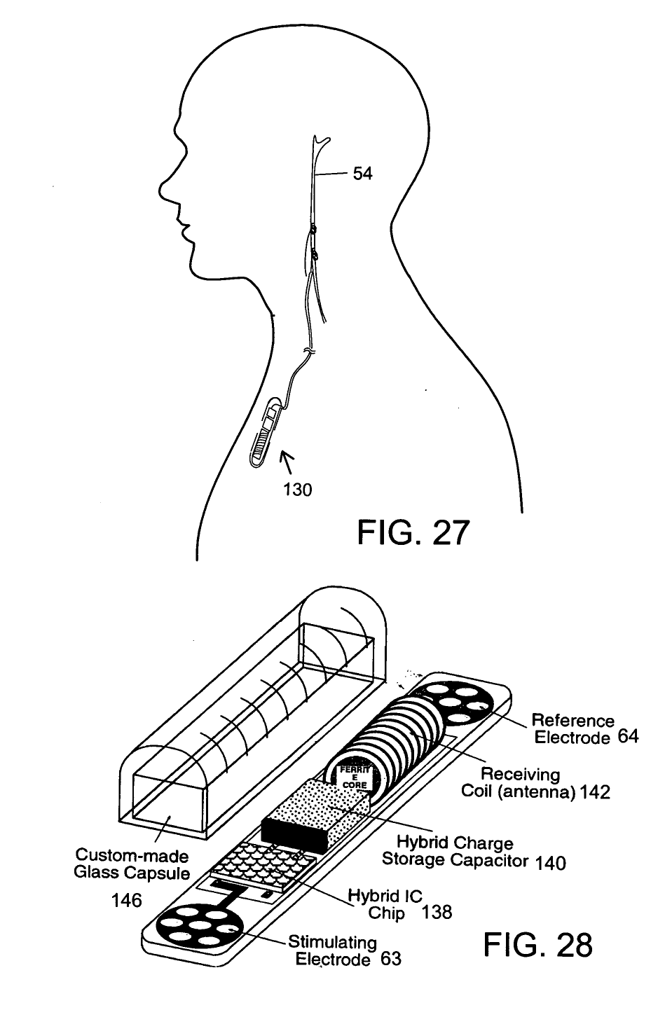

In one embodiment, a microstimulator 130 may be used for providing pulses to the vagus nerve(s) 54. Shown in conjunction with FIG. 26A, is a microstimulator where the electrical circuitry 132 and power source 134 are encased in a miniature hermetically sealed enclosure, and only the electrodes 63, 64 are exposed. FIG. 26B depicts the same microstimulator, except the electrodes are modified and adapted to wrap around the nerve tissue 54. Because of its small size, the whole microstimulator may be in the proximity of the nerve tissue to be stimulated, or alternatively as shown in conjunction with FIG. 27, the microstimulator may be implanted at a different site, and connected to the electrodes via conductors insulated with silicone and polyurethane (FIG. 26C).

Shown in reference with FIG. 28 is the overall structure of an implantable microstimulator 130. It consists of a micromachined silicon substrate that incorporates two stimulating electrodes which are the cathode and anode of a bipolar stimulating electrode pair 63, 64; a hybrid-connected tantalum chip capacitor 140 for power storage; a receiving coil 142; a bipolar-CMOS integrated circuit chip 138 for power regulation and control of the microstimulator; and a custom made glass capsule 146 that is electrostatically bonded to the silicon carrier to provide a hermetic package for the receiver-stimulator circuitry and hybrid elements. The stimulating electrode pair 63,64 resides outside of the package and feedthroughs are used to connect the internal electronics to the electrodes.

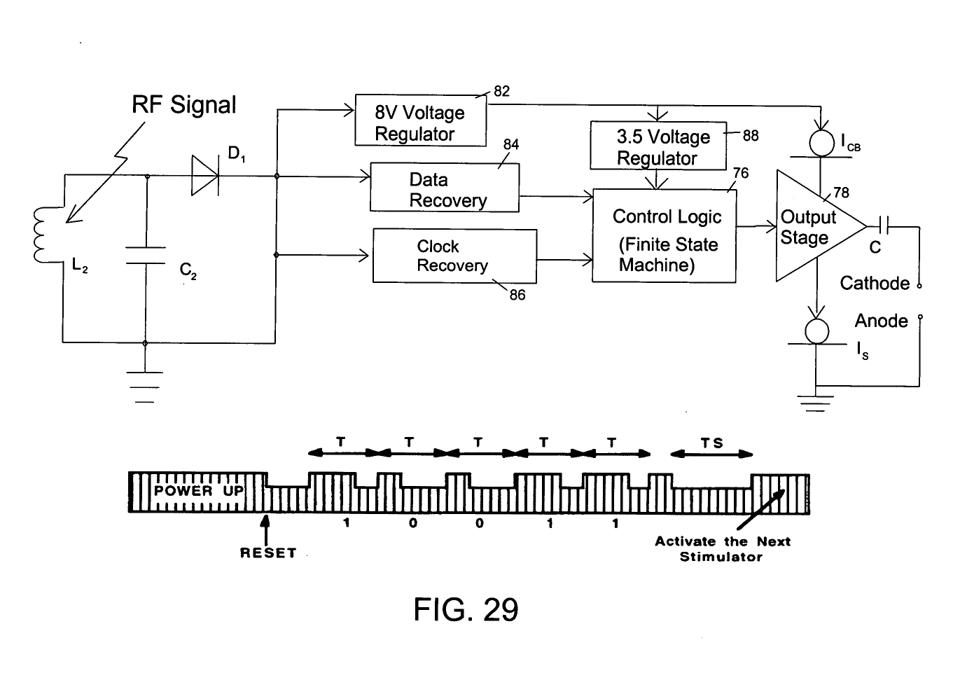

FIG. 29 shows the overall system electronics required for the microstimulator, and the power and data transmission protocol used for radiofrequency telemetry. The circuit receives an amplitude modulated RF carrier from an external transmitter and generates 8-V and 4-V dc supplies, generates a clock from the carrier signal, decodes the modulated control data, interprets the control data, and generates a constant current output pulse when appropriate. The RF carrier used for the telemetry link has a nominal frequency of around 1.8 MHz, and is amplitude modulated to encode control data. Logical “1” and “0” are encoded by varying the width of the amplitude modulated carrier, as shown in the bottom portion of FIG. 29. The carrier signal is initially high when the transmitter is turned on and sets up an electromagnetic field inside the transmitter coil. The energy in the field is picked up by receiver coils 142, and is used to charge the hybrid capacitor 140. The carrier signal is turned high and then back down again, and is maintained at the low level for a period between 1-200 μsec. The microstimulator 130 will then deliver a constant current pulse into the nerve tissue through the stimulating electrode pair 63, 64 for the period that the carrier is low. Finally, the carrier is turned back high again, which will indicate the end of the stimulation period to the microstimulator 130, thus allowing it to charge its capacitor 140 back up to the on-chip voltage supply.

On-chip circuitry has been designed to generate two regulated power supply voltages (4V and 8V) from the RF carrier, to demodulate the RF carrier in order to recover the control data that is used to program the microstimulator, to generate the clock used by the on-chip control circuitry, to deliver a constant current through a controlled current driver into the nerve tissue, and to control the operation of the overall circuitry using a low-power CMOS logic controller.

### Programmable Implantable Pulse Generator (IPG)

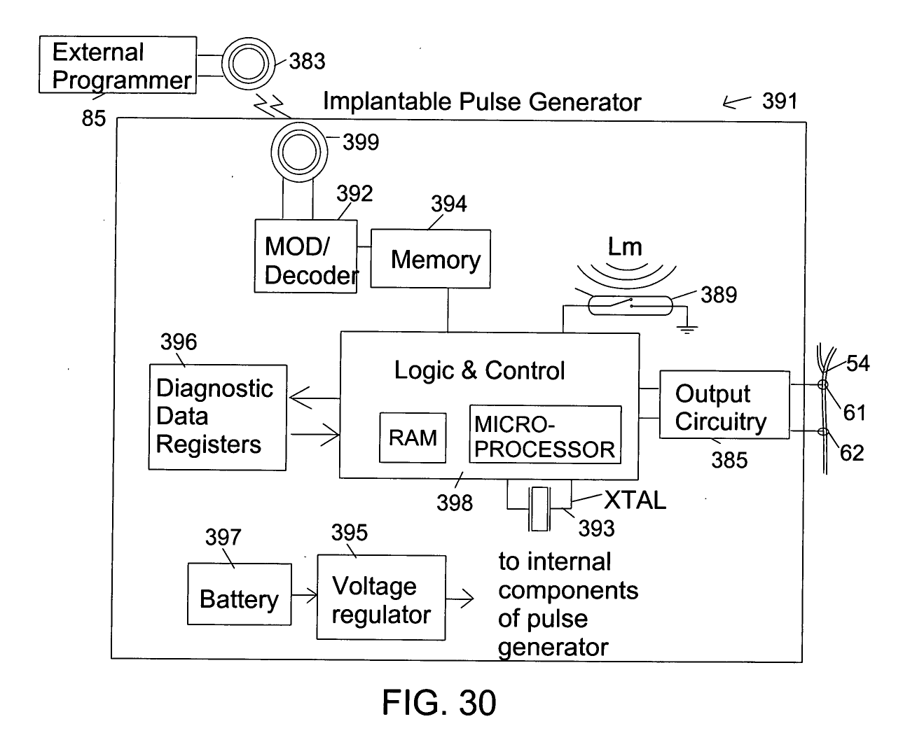

In one embodiment, a fully programmable implantable pulse generator (IPG) may be used. Shown in conjunction with FIG. 30, the implantable pulse generator unit 391 is preferably a microprocessor based device, where the entire circuitry is encased in a hermetically sealed titanium can. As shown in the overall block diagram, the logic & control unit 398 provides the proper timing for the output circuitry 385 to generate electrical pulses that are delivered to electrodes 61, 62 via a lead 40 (not shown). Programming of the implantable pulse generator (IPG) 391 is done via an external programmer 85. Once programmed via an external programmer 85, the implanted pulse generator 391 provides appropriate electrical stimulation pulses to the vagus nerve(s) 54 via electrodes 61,62.

This embodiment may also comprise optional fixed pre-determined/pre-packaged programs. Examples of LOW, LOW-MED, MED, and HIGH stimulation states were given in the previous section, under “Programmer-less Implantable Pulse Generator (IPG)”. These pre-packaged/pre-determined programs comprise unique combinations of pulse amplitude, pulse width, pulse frequency, ON-time and OFF-time. Advantageously, a number of these “pre-determined/pre-packaged programs” may be stored in a “library”, and activated in a simple fashion, without having to program each parameter individually.

In addition, each parameter may be individually programmed and stored in memory. The range of programmable electrical stimulation parameters are shown in table 3 below.

TABLE 3
Programmable electrical parameter range

| PARAMER | RANGE |
|---|---|
| Pulse Amplitude | 0.1 Volt-15 Volts |
| Pulse width | 20 μS-5 mSec. |
| Frequency | 3 Hz-300 Hz |
| On-time | 5 Secs-24 hours |
| Off-time | 5 Secs-24 hours |
| Ramp | ON/OFF |

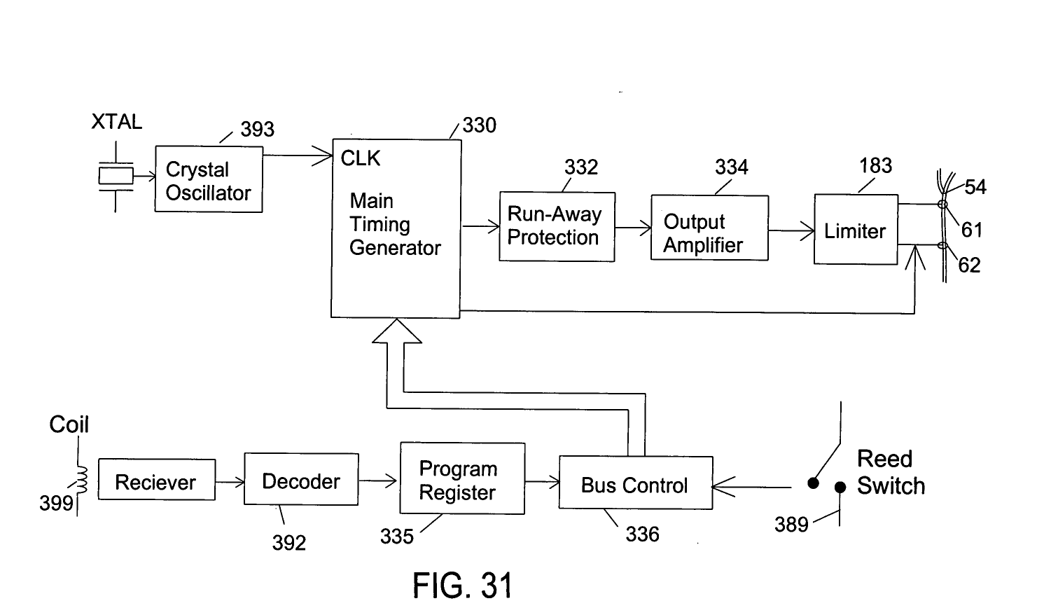

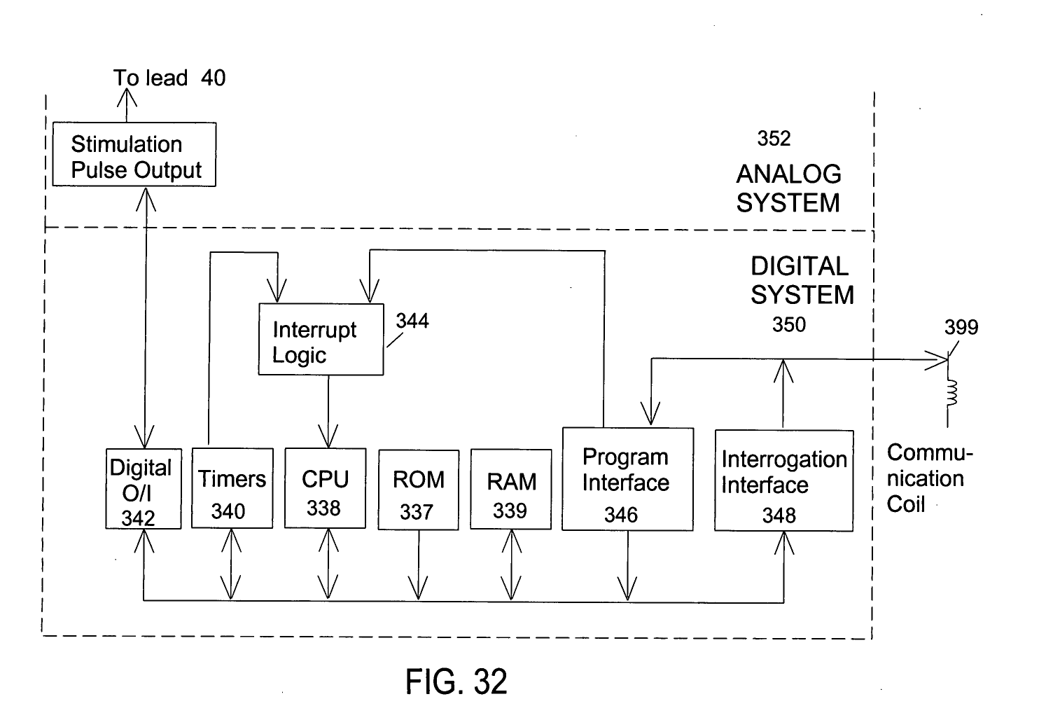

Shown in conjunction with FIGS. 31 and 32, the electronic stimulation module comprises both digital 350 and analog 352 circuits. A main timing generator 330 (shown in FIG. 31), controls the timing of the analog output circuitry for delivering neuromodulating pulses to the vagus nerve(s) 54, via output amplifier 334. Limiter 183 prevents excessive stimulation energy from getting into the vagus nerve(s) 54. The main timing generator 330 receiving clock pulses from crystal oscillator 393. Main timing generator 330 also receiving input from programmer 85 via coil 399. FIG. 32 highlights other portions of the digital system such as CPU 338, ROM 337, RAM 339, program interface 346, interrogation interface 348, timers 340, and digital O/I 342. The functioning details of these circuits is well known to one skilled in the art.

Most of the digital functional circuitry 350 is on a single chip (IC). This monolithic chip along with other IC's and components such as capacitors and the input protection diodes are assembled together on a hybrid circuit. As well known in the art, hybrid technology is used to establish the connections between the circuit and the other passive components. The integrated circuit is hermetically encapsulated in a chip carrier. A coil 399 connected to the hybrid is used for bidirectional telemetry. The hybrid and battery 397 are encased in a titanium can. This housing is a two-part titanium capsule that is hermetically sealed by laser welding. Alternatively, electron-beam welding can also be used. The header 79 is a cast epoxy-resin with hermetically sealed feed-through, and form the lead 40 connection block.

### Combination Implantable Device Comprising Both a Stimulus-Receiver and a Programmable Implantable Pulse Generator (IPG)

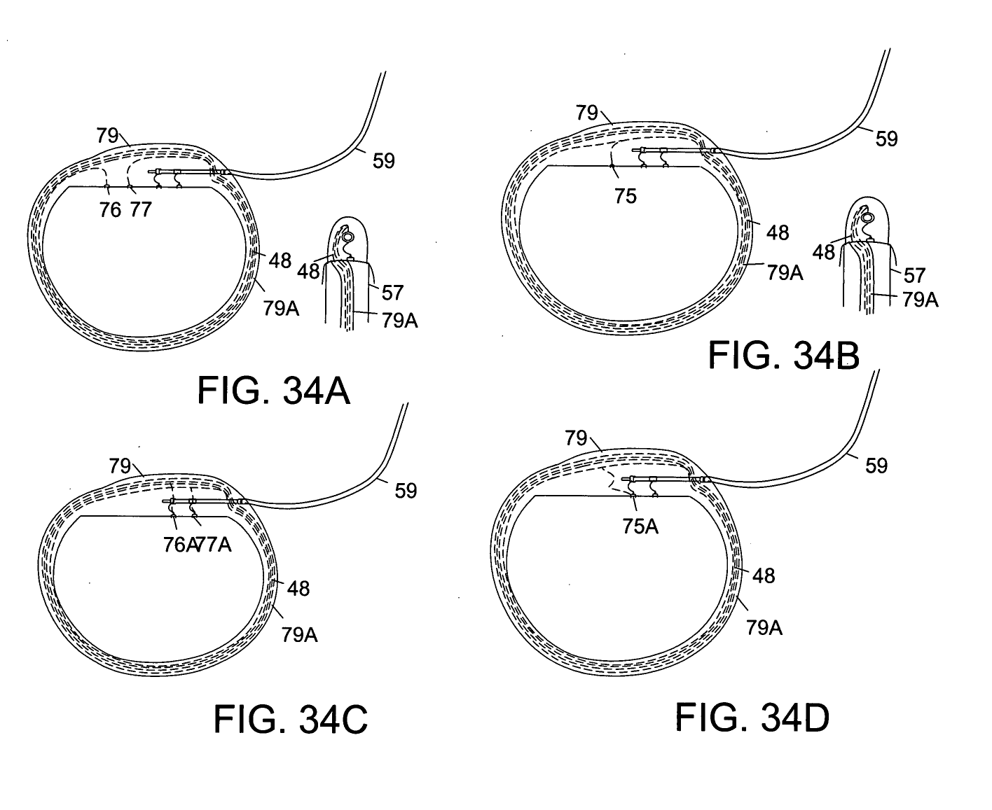

In one embodiment, the implantable device may comprise both a stimulus-receiver and a programmable implantable pulse generator (IPG) in one device. FIG. 33 shows a close up view of the packaging of the implanted stimulator 75 of this embodiment, showing the two subassemblies 120, 70. The two subassemblies are the stimulus-receiver module 120 and the battery operated pulse generator module 70. The electrical components of the stimulus-receiver module 120 may be substantially in the titanium case along with other circuitry, except for a coil. The coil may be outside the titanium case as shown in FIG. 33, or the coil 48C may be externalized at the header portion 79C of the implanted device, and may be wrapped around the titanium can. In this case, the coil is encased in the same material as the header 79C, as shown in FIGS. 34A-D. FIG. 34A depicts a bipolar configuration with two separate feed-throughs, 76, 77. FIG. 34B depicts a unipolar configuration with one separate feed-through. FIG. 34C, and 34D depict the same configuration except the feed-throughs are common with the feed-throughs for the lead.

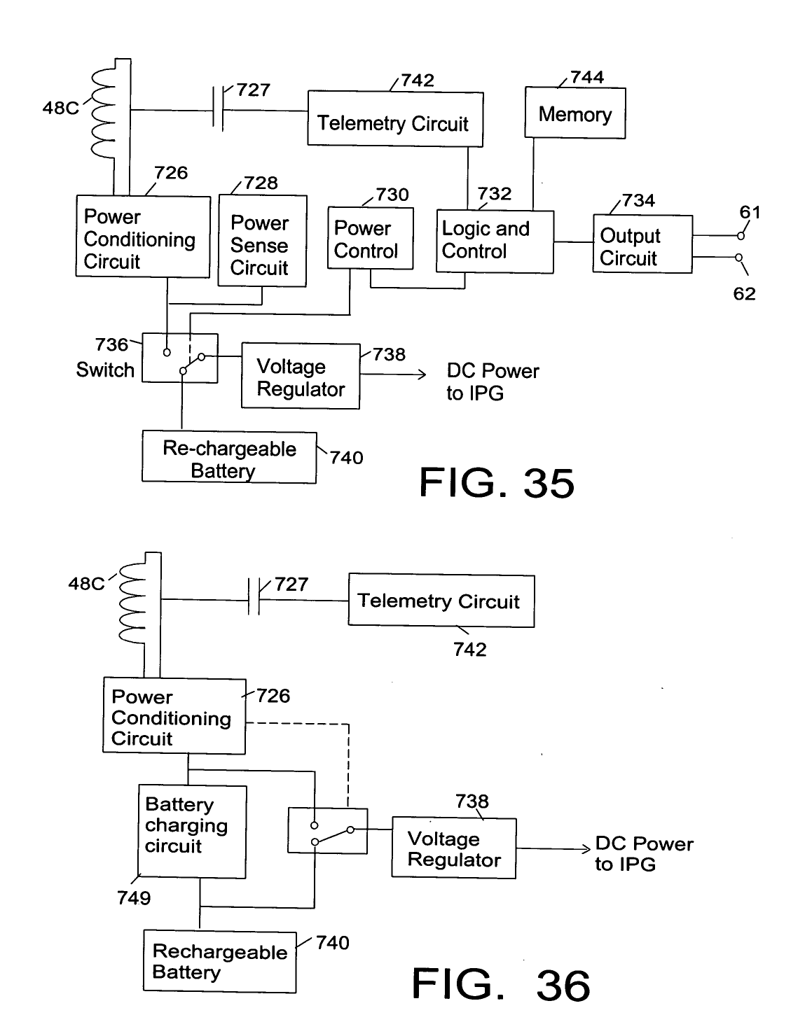

FIG. 35 is a simplified overall block diagram of the embodiment where the implanted stimulator 75 is a combination device, which may be used as a stimulus-receiver (SR) in conjunction with an external stimulator, or the same implanted device may be used as a traditional programmable implanted pulse generator (IPG). The coil 48C which is external to the titanium case may be used both as a secondary of a stimulus-receiver, or may also be used as the forward and back telemetry coil.

In this embodiment, as disclosed in FIG. 35, the IPG circuitry within the titanium case is used for all stimulation pulses whether the energy source is the internal battery 740 or an external power source. The external device serves as a source of energy, and as a programmer that sends telemetry to the IPG. For programming, the energy is sent as high frequency sine waves with superimposed telemetry wave driving the external coil 46C. Once received by the implanted coil 48C, the telemetry is passed through coupling capacitor 727 to the IPG's telemetry circuit 742. For pulse delivery using external power source, the stimulus-receiver portion will receive the energy coupled to the implanted coil 48C and, using the power conditioning circuit 726, rectify it to produce DC, filter and regulate the DC, and couple it to the IPG's voltage regulator 738 section so that the IPG can run from the externally supplied energy rather than the implanted battery 740.

The system provides a power sense circuit 728 that senses the presence of external power communicated with the power control 730 when adequate and stable power is available from an external source. The power control circuit controls a switch 736 that selects either battery power 740 or conditioned external power from 726. The logic and control section 732 and memory 744 includes the IPG's microcontroller, pre-programmed instructions, and stored chagneable parameters. Using input for the telemetry circuit 742 and power control 730, this section controls the output circuit 734 that generates the output pulses.

It will be clear to one skilled in the art that this embodiment of the invention can also be practiced with a rechargeable battery. This version is shown in conjunction with FIG. 36. The circuitry in the two versions are similar except for the battery charging circuitry 749. This circuit is energized when external power is available. It senses the charge state of the battery and provides appropriate charge current to safely recharge the battery without overcharging.

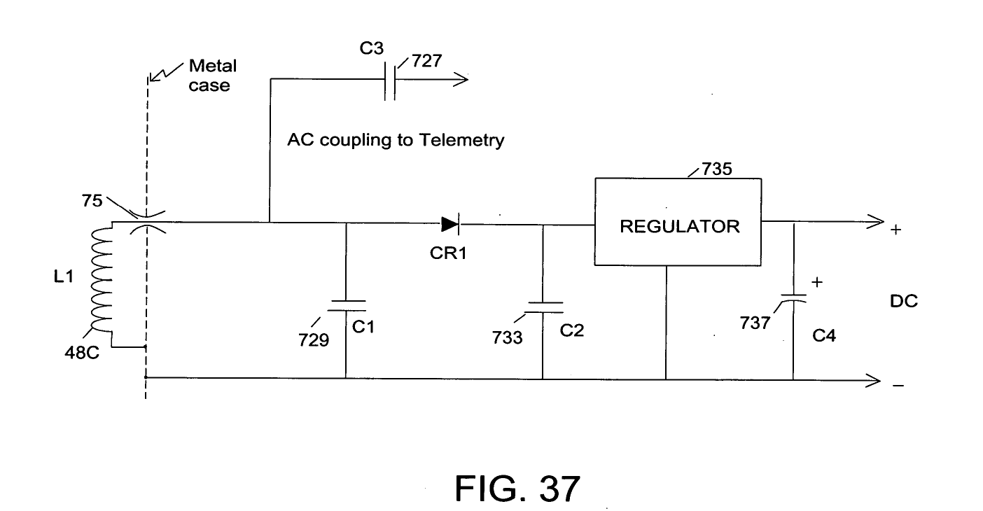

The stimulus-receiver portion of the circuitry is shown in conjunction with FIG. 37. Capacitor C1 (729) makes the combination of C1 and L1 sensitive to the resonant frequency and less sensitive to other frequencies, and energy from an external (primary) coil 46C is inductively transferred to the implanted unit via the secondary coil 48C. The AC signal is rectified to DC via diode 731, and filtered via capacitor 733. A regulator 735 sets the output voltage and limits it to a value just above the maximum IPG cell voltage. The output capacitor C4 (737), typically a tantalum capacitor with a value of 100 micro-Farads or greater, stores charge so that the circuit can supply the IPG with high values of current for a short time duration with minimal voltage change during a pulse while the current draw from the external source remains relatively constant. Also shown in conjunction with FIG. 37, a capacitor C3 (727) couples signals for forward and back telemetry.

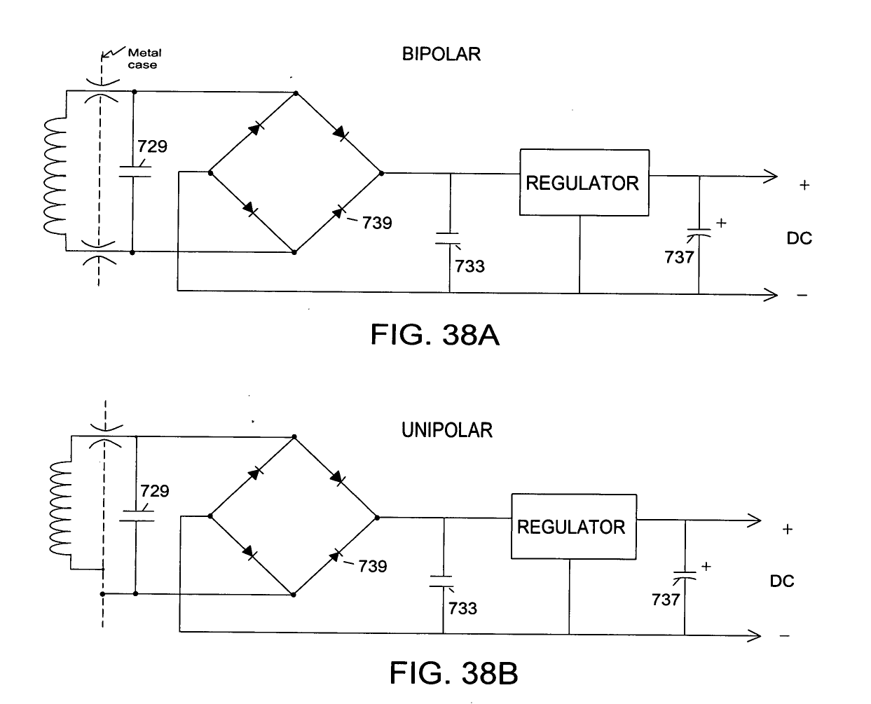

FIGS. 38A and 38B show alternate connection of the receiveing coil. In FIG. 38A, each end of the coil is connected to the circuit through a hermetic feedthrough filter. In this instance, the DC output is floating with respect to the IPG's case. In FIG. 38B, one end of the coil is connected to the exterior of the IPG's case. The circuit is completed by connecting the capacitor 729 and bridge rectifier 739 to the interior of the IPG's case The advantage of this arrangement is that it requires one less hermetic feedthrough filter, thus reducing the cost and improving the reliabilty of the IPG. Hermetic feedthrough filters are expensive and a possible failure point. However, the case connection may complicit the output circuitry or limit its versatility. When using a bipolar electrode, care must be taken to prevent an unwanted return path for the pulse to the IPG's case. This is not a concern for unipolar pulses using a single conductor electrode because it relies on the IPG's case a return for the pulse current.

In the unipolar configuration, advantageously a bigger tissue area is stimulated since the difference between the tip (cathode) and case (anode) is larger. Stimulation using both configuration is considered within the scope of this invention.

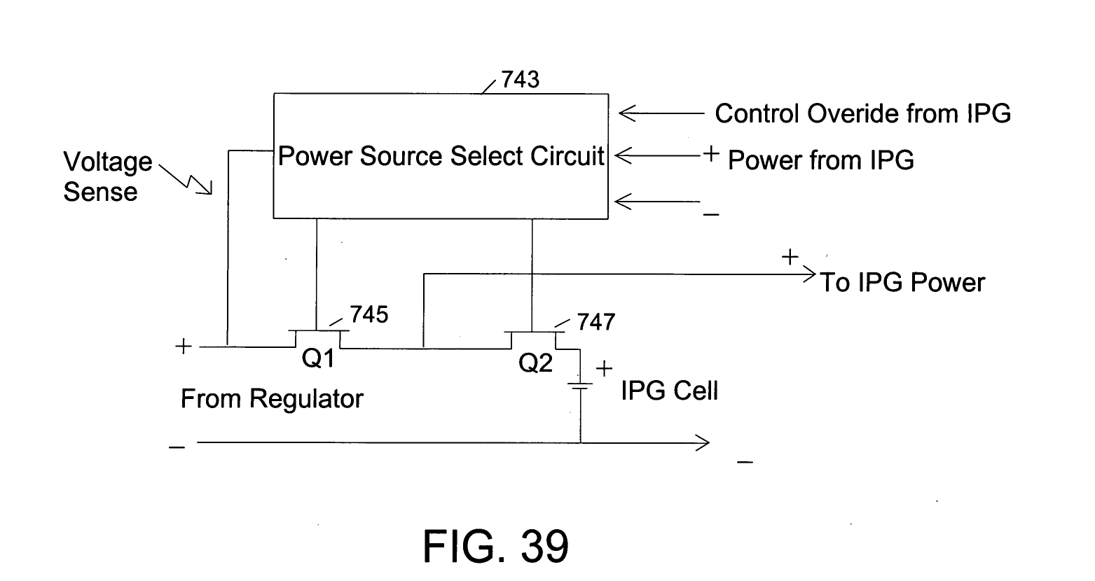

The power source select circuit is highlighted in conjunction with FIG. 39. In this embodiment, the IPG provides stimulation pulses according to the stimulation programs stored in the memory 744 of the implanted stimulator, with power being supplied by the implanted battery 740. When stimulation energy from an external stimulator is inductively received via secondary coil 48C, the power source select circuit (shown in block 743) switches power via transistor Q1 745 and transistor Q2 743. Transistor Q1 and Q2 are preferably low loss MOS transistor used as switches, even though other types of transistors may be used.

### Implantable Pulse Generator (IPG) Comprising a Rechargable Battery

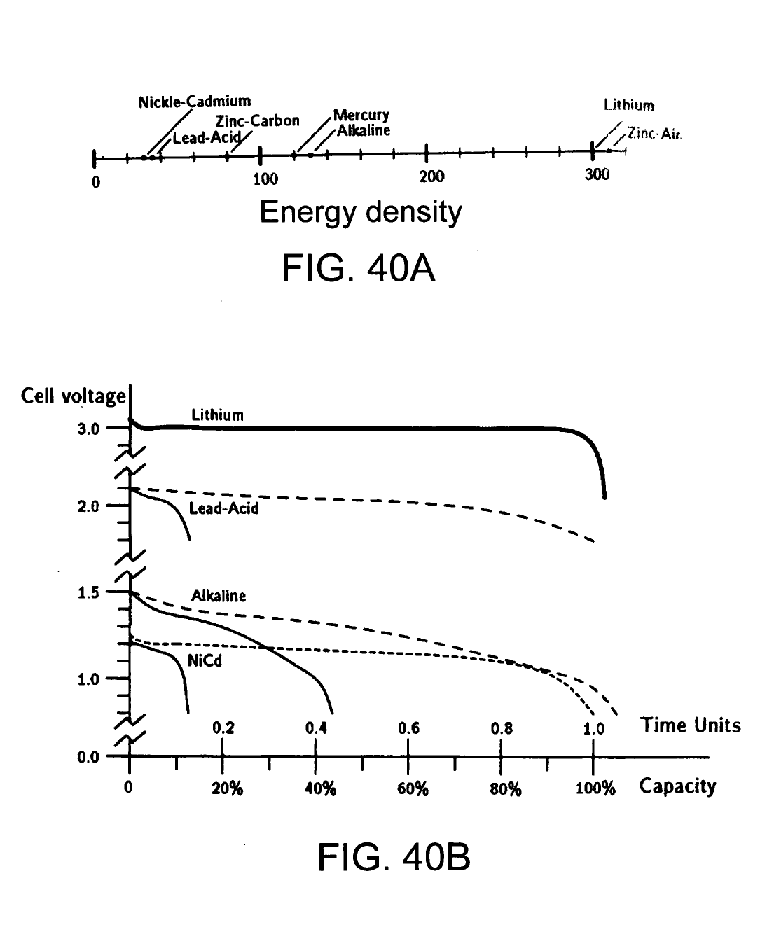

In one embodiment, an implantable pulse generator with rechargeable power source can be used. Because of the rapidity of the pulses required for modulating nerve tissue 54 (unlike cardiac pacing), there is a real need for power sources that will provide an acceptable service life under conditions of continuous delivery of high frequency pulses. FIG. 40A shows a graph of the energy density of several commonly used battery technologies. Lithium batteries have by far the highest energy density of commonly available batteries. Also, a lithium battery maintains a nearly constant voltage during discharge. This is shown in conjunction with FIG. 40B, which is normalized to the performance of the lithium battery. Lithium-ion batteries also have a long cycle life, and no memory effect. However, Lithium-ion batteries are not as tolerant to overcharging and overdischarging. One of the most recent development in rechargable battery technology is the Lithium-ion polymer battery. Recently the major battery manufacturers (Sony, Panasonic, Sanyo) have announced plans for Lithium-ion polymer battery production.

In another embodiment, existing nerve stimulators and cardiac pacemakers can be modified to incorporate rechargeable batteries. Among the nerve stimulators that can be adopted with rechargeable batteries can for, example, be the vagus nerve stimulator manufactured by Cyberonics Inc. (Houston, Tex.). U.S. Pat. No. 4,702,254 (Zabara), U.S. Pat. No. 5,023,807 (Zabara), and U.S. Pat. No, 4,867,164 (Zabara) on Neurocybernetic Prostheses, which can be practiced with rechargeable power source as disclosed in the next section. These patents are incorporated herein by reference.

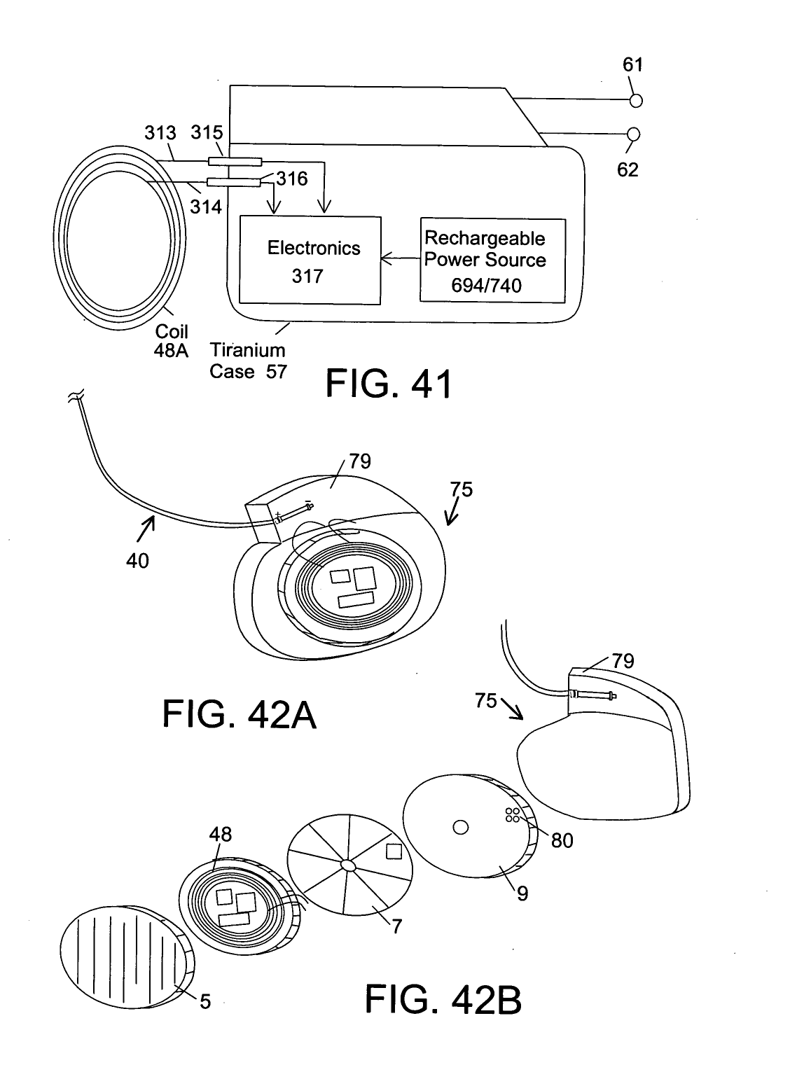

As shown in conjunction with FIG. 41, the coil is externalized from the titanium case 57. The RF pulses transmitted via coil 46 and received via subcutaneous coil 48A are rectified via a diode bridge. These DC pulses are processed and the resulting current applied to recharge the battery 694/740 in the implanted pulse generator. In one embodiment the coil 48C may be externalized at the header portion 79 of the implanted device, and may be wrapped around the titanium can, as was previously shown in FIGS. 34A-D.

In one embodiment, the coil may also be positioned on the titanium case as shown in conjunction with FIGS. 42A and 42B. FIG. 42A shows a diagram of the finished implantable stimulator 391 R of one embodiment. FIG. 42B shows the pulse generator with some of the components used in assembly in an exploded view. These components include a coil cover 7, the secondary coil 48 and associated components, a magnetic shield 9, and a coil assembly carrier 11. The coil assembly carrier 11 has at least one positioning detail 13 located between the coil assembly and the feed through for positioning the electrical connection. The positioning detail 13 secures the electrical connection.

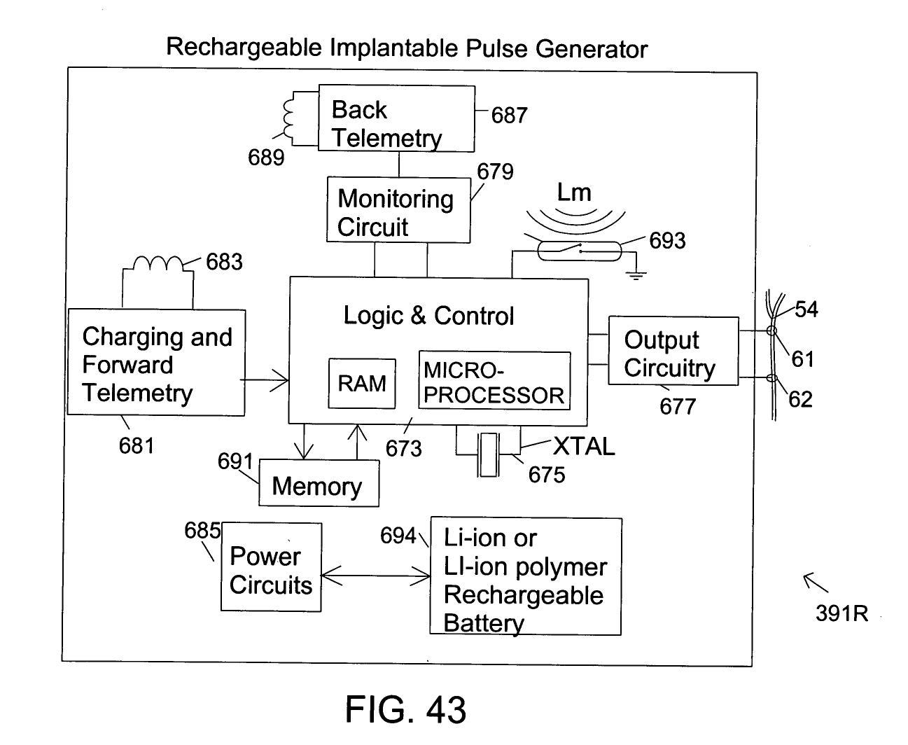

A schematic diagram of the implanted pulse generator (IPG 391R), with rechargeable battery 694, is shown in conjunction with FIG. 43. The IPG 391R includes logic and control circuitry 673 connected to memory circuitry 691. The operating program and stimulation parameters are typically stored within the memory 691 via forward telemetry. Stimulation pulses are provided to the nerve tissue 54 via output circuitry 677 controlled by the microcontroller.

The operating power for the IPG 391R is derived from a rechargeable power source 694. The rechargeable power source 694 comprises a rechargeable lithium-ion or lithium-ion polymer battery. Recharging occurs inductively from an external charger to an implanted coil 48B underneath the skin 60. The rechargeable battery 694 may be recharged repeatedly as needed. Additionally, the IPG 391R is able to monitor and telemeter the status of its rechargable battery 691 each time a communication link is established with the external programmer 85.

Much of the circuitry included within the IPG 391R may be realized on a single application specific integrated circuit (ASIC). This allows the overall size of the IPG 391R to be quite small, and readily housed within a suitable hermetically-sealed case. The IPG case is preferably made from a titanium and is shaped in a rounded case.

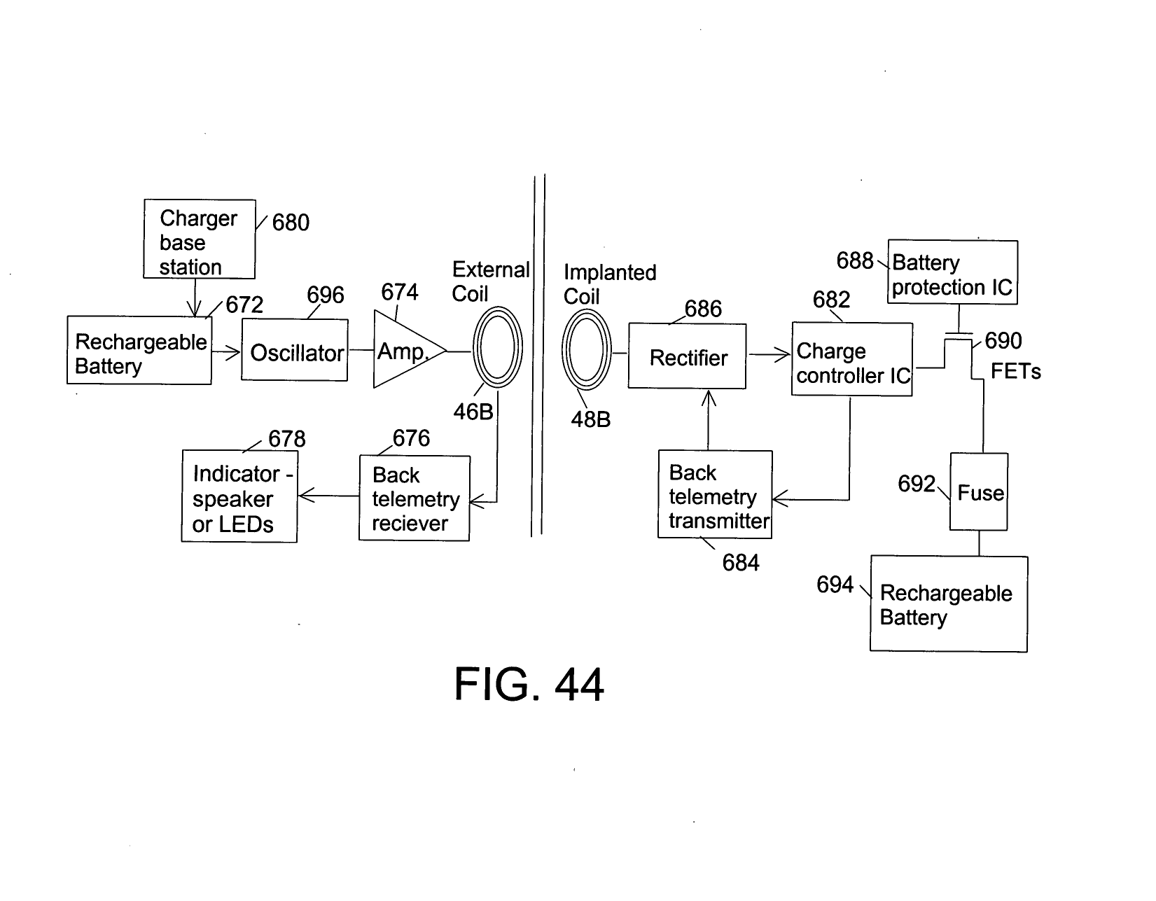

Shown in conjunction with FIG. 44 are the recharging elements of this embodiment. The re-charging system uses a portable external charger to couple energy into the power source of the IPG 391R. The DC-to-AC conversion circuitry 696 of the re-charger receives energy from a battery 672 in the re-charger. A charger base station 680 and conventional AC power line may also be used. The AC signals amplified via power amplifier 674 are inductively coupled between an external coil 46B and an implanted coil 48B located subcutaneously with the implanted pulse generator (IPG) 391R. The AC signal received via implanted coil 48B is rectified 686 to a DC signal which is used for recharging the rechargeable battery 694 of the IPG, through a charge controller IC 682. Additional circuitry within the IPG 391R includes, battery protection IC 688 which controls a FET switch 690 to make sure that the rechargeable battery 694 is charged at the proper rate, and is not overcharged. The battery protection IC 688 can be an off-the-shelf IC available from Motorola (part no. MC 33349N-3R1). This IC monitors the voltage and current of the implanted rechargeable battery 694 to ensure safe operation. If the battery voltage rises above a safe maximum voltage, the battery protection IC 688 opens charge enabling FET switches 690, and prevents further charging. A fuse 692 acts as an additional safeguard, and disconnects the battery 694 if the battery charging current exceeds a safe level. As also shown in FIG. 44, charge completion detection is achieved by a back-telemetry transmitter 684, which modulates the secondary load by changing the full-wave rectifier into a half-wave rectifier/voltage clamp. This modulation is in turn, sensed by the charger as a change in the coil voltage due to the change in the reflected impedance. When detected through a back telemetry receiver 676, either an audible alarm is generated or a LED is turned on.

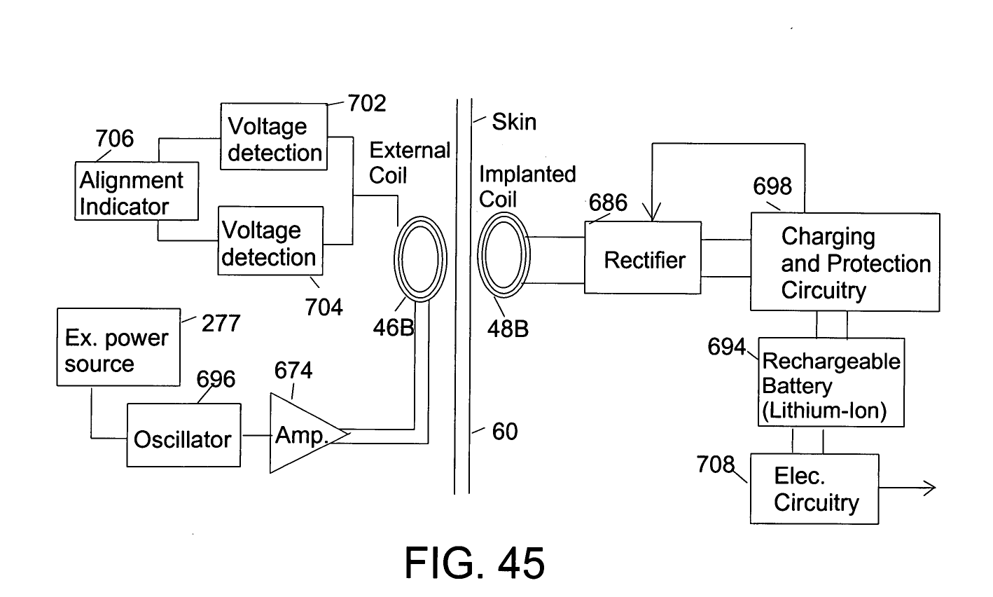

A simplified block diagram of charge completion and misalignment detection circuitry is shown in conjunction with FIG. 45. As shown, a switch regulator 686 operates as either a full-wave rectifier circuit or a half-wave rectifier circuit as controlled by a control signal (CS) generated by charging and protection circuitry 698. The energy induced in implanted coil 48B (from external coil 46B) passes through the switch rectifier 686 and charging and protection circuitry 698 to the implanted rechargeable battery 694. As the implanted battery 694 continues to be charged, the charging and protection circuitry 698 continuously monitors the charge current and battery voltage. When the charge current and battery voltage reach a predetermined level, the charging and protection circuitry 698 triggers a control signal. This control signal causes the switch rectifier 686 to switch to half-wave rectifier operation. When this change happens, the voltage sensed by voltage detector 702 causes the alignment indicator 706 to be activated. This indicator 706 may be an audible sound or a flashing LED type of indicator.

The indicator 706 may similarly be used as a misalignment indicator. In normal operation, when coils 46B (external) and 48B (implanted) are properly aligned, the voltage Vₛ sensed by voltage detector 704 is at a minimum level because maximum energy transfer is taking place. If and when the coils 46B and 48B become misaligned, then less than a maximum energy transfer occurs, and the voltage Vₛ sensed by detection circuit 704 increases significantly. If the voltage Vₛ reaches a predetermined level, alignment indicator 706 is activated via an audible speaker and/or LEDs for visual feedback. After adjustment, when an optimum energy transfer condition is established, causing Vₛ to decrease below the predetermined threshold level, the alignment indicator 706 is turned off.

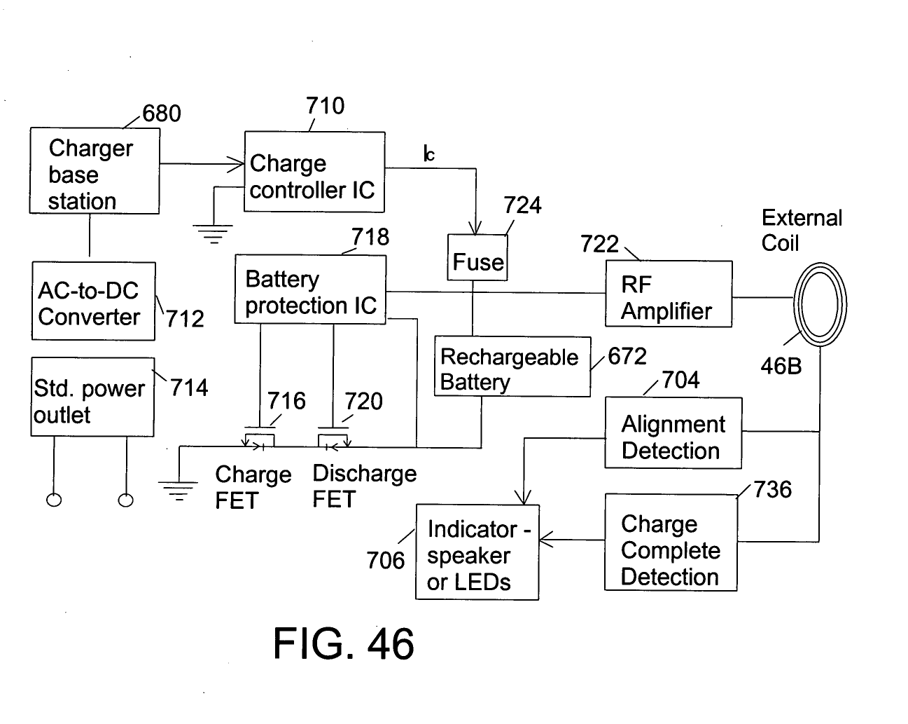

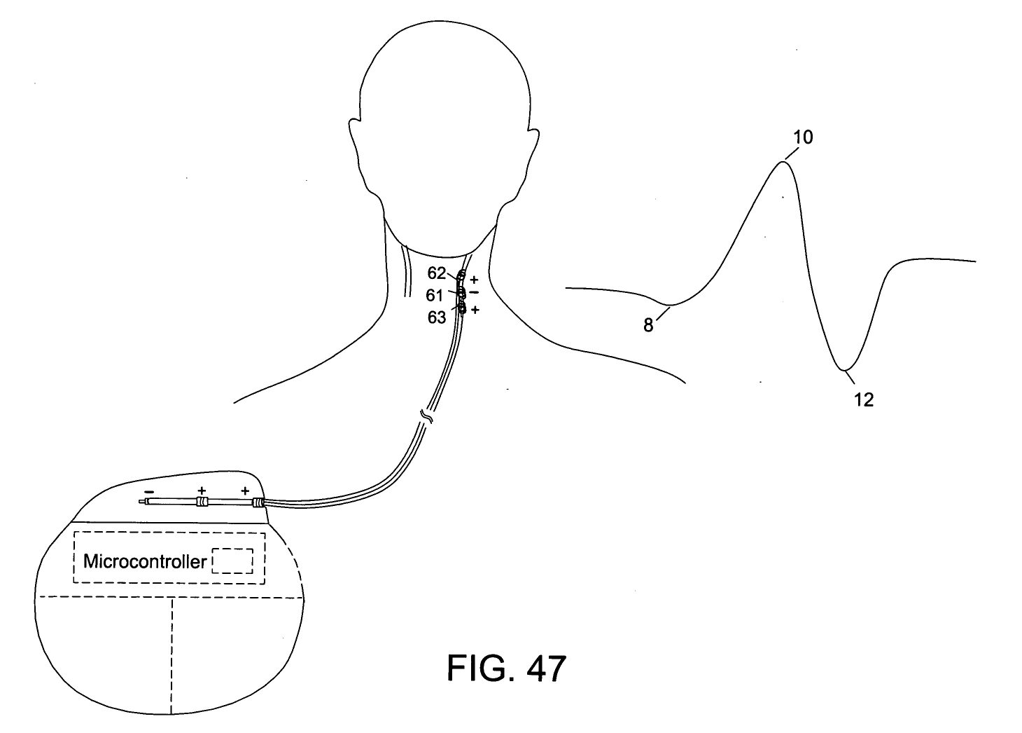

The elements of the external recharger are shown as a block diagram in conjunction with FIG. 46. In this disclosure, the words charger and recharger are used interchangeably. The charger base station 680 receives its energy from a standard power outlet 714, which is then converted to 5 volts DC by a AC-to-DC transformer 712. When the re-charger is placed in a charger base station 680, the re-chargeable battery 672 of the re-charger is fully recharged in a few hours and is able to recharge the battery 694 of the IPG 391R. If the battery 672 of the external re-charger falls below a prescribed limit of 2.5 volt DC, the battery 672 is trickle charged until the voltage is above the prescribed limit, and then at that point resumes a normal charging process.

As also shown in FIG. 46, a battery protection circuit 718 monitors the voltage condition, and disconnects the battery 672 through one of the FET switches 716, 720 if a fault occurs until a normal condition returns. A fuse 724 will disconnect the battery 672 should the charging or discharging current exceed a prescribed amount.

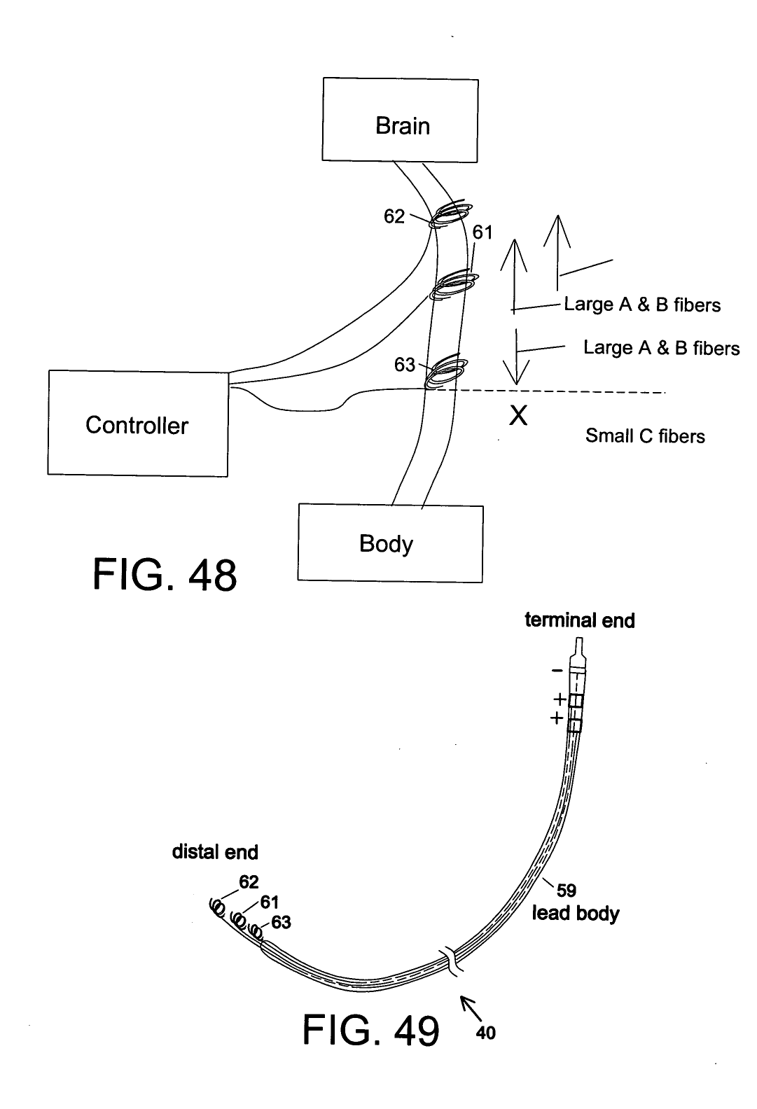

Since another key concept of this invention is to deliver afferent stimulation to vagus nerve(s), in one aspect efferent stimulation of selected types of fibers may be substantially blocked, utilizing the “greenwave” effect. In such a case, as shown in conjunction with FIGS. 47 and 48, a tripolar lead is utilized. As depicted on the top right portion of FIG. 47, there is a depolarization peak 10 on the vagus nerve bundle corresponding to electrode 61 (cathode) and the two hyper-polarization peaks 8, 12 corresponding to electrodes 62, 63 (anodes). With the microcontroller controlling the tripolar device, the size and timing of the hyper-polarizations 8, 12 can be controlled. Since the speed of conduction is different between the larger diameter A and B fibers and the smaller diameter c-fibers, by appropriately timing the pulses, collision blocks can be created for conduction via the large diameter A and B fibers in the efferent direction. This is depicted schematically in FIG. 48. A number of blocking techniques are known in the art, such as collision blocking, high frequency blocking, and anodal blocking. Any of these well known blocking techniques may be used with the practice of this invention, and are considered within the scope of this invention. A lead with tripolar electrodes for stimulation/blocking is shown in conjunction with FIG. 49.

In summary, in the method of the current invention for neuromodulation of cranial nerve such as the vagus nerve(s), to provide adjunct therapy along with rTMS for psychiatric disorders, neuropsychiatric disorders and cognitive impairments, can be practiced with any of the several pulse generator systems disclosed including,
a) an implanted stimulus-receiver with an external stimulator;
b) an implanted stimulus-receiver comprising a high value capacitor for storing charge, used in conjunction with an external stimulator;
c) a programmer-less implantable pulse generator (IPG) which is operable with a magnet;
d) a microstimulator;
e) a programmable implantable pulse generator;
f) a combination implantable device comprising both a stimulus-receiver and a programmable IPG; and
g) an IPG comprising a rechargeable battery.

Neuromodulation of vagus nerve(s) with any of these systems is considered within the scope of this invention.

In one embodiment, the external stimulator and/or the programmer has a telecommunications module, as described in a co-pending application, and summarized here for reader convenience. The telecommunications module has two-way communications capabilities.

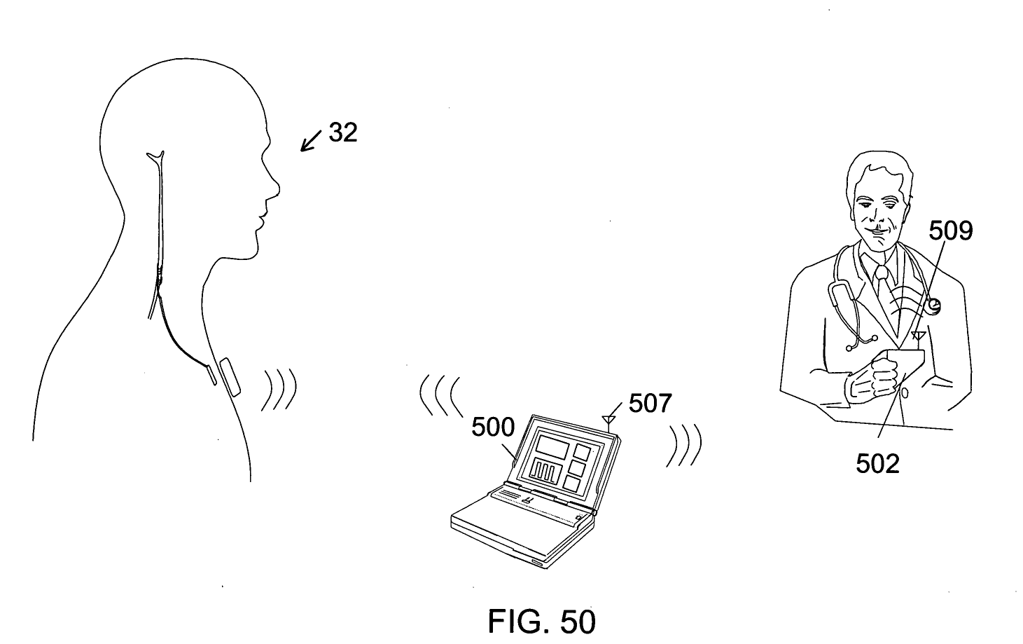

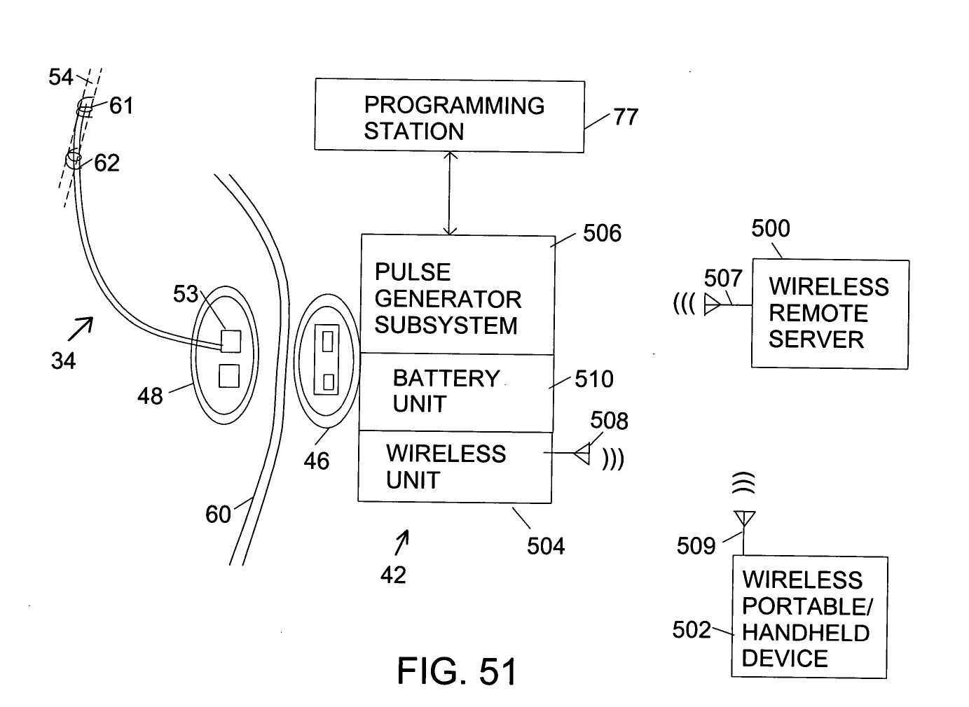

FIGS. 50 and 51 depict communication between an external stimulator 42 and a remote hand-held computer 502. A desktop or laptop computer can be a server 500 which is situated remotely, perhaps at a physician's office or a hospital. The stimulation parameter data can be viewed at this facility or reviewed remotely by medical personnel on a hand-held personal data assistant (PDA) 502, such as a “palm-pilot” from PALM corp. (Santa Clara, Calif.), a “Visor” from Handspring Corp. (Mountain view, Calif.) or on a personal computer (PC). The physician or appropriate medical personnel, is able to interrogate the external stimulator 42 device and know what the device is currently programmed to, as well as, get a graphical display of the pulse train. The wireless communication with the remote server 500 and hand-held PDA 502 would be supported in all geographical locations within and outside the United States (US) that provides cell phone voice and data communication service.

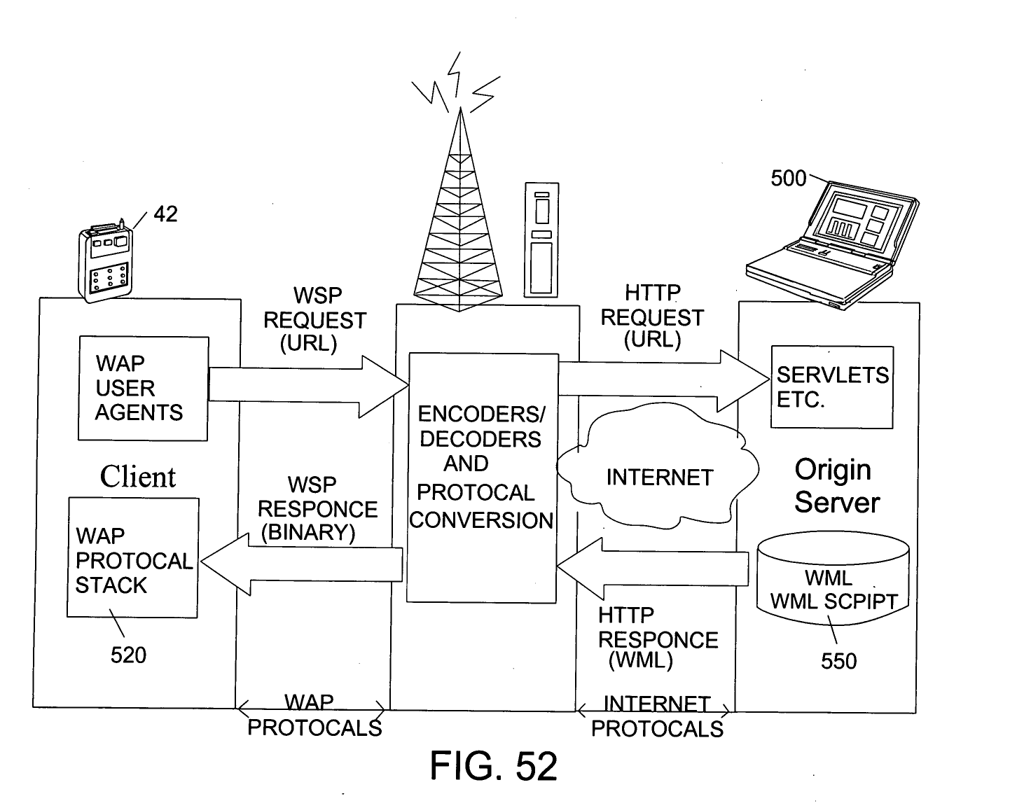

In one aspect of the invention, the telecommunications component can use Wireless Application Protocol (WAP). The Wireless Application Protocol (WAP), which is a set of communication protocols standardizing Internet access for wireless devices. While previously, manufacturers used different technologies to get Internet on hand-held devices, with WAP devices and services interoperate. WAP also promotes convergence of wireless data and the Internet. The WAP programming model is heavily based on the existing Internet programming model, and is shown schematically in FIG. 52. Introducing a gateway function provides a mechanism for optimizing and extending this model to match the characteristics of the wireless environment. Over-the-air traffic is minimized by binary encoding/decoding of Web pages and readapting the Internet Protocol stack to accommodate the unique characteristics of a wireless medium such as call drops.

The key components of the WAP technology, as shown in FIG. 52, includes 1) Wireless Mark-up Language (WML) 550 which incorporates the concept of cards and decks, where a card is a single unit of interaction with the user. A service constitutes a number of cards collected in a deck. A card can be displayed on a small screen. WML supported Web pages reside on traditional Web servers. 2) WML Script which is a scripting language, enables application modules or applets to be dynamically transmitted to the client device and allows the user interaction with these applets. 3) Microbrowser, which is a lightweight application resident on the wireless terminal that controls the user interface and interprets the WML/WMLScript content. 4) A lightweight protocol stack 520 which minimizes bandwidth requirements, guaranteeing that a broad range of wireless networks can run WAP applications. The protocol stack of WAP can comprise a set of protocols for the transport (WTP), session (WSP), and security (WTLS) layers. WSP is binary encoded and able to support header caching, thereby economizing on bandwidth requirements. WSP also compensates for high latency by allowing requests and responses to be handled asynchronously, sending before receiving the response to an earlier request. For lost data segments, perhaps due to fading or lack of coverage, WTP only retransmits lost segments using selective retransmission, thereby compensating for a less stable connection in wireless. The above mentioned features are industry standards adopted for wireless applications and greater details have been publicized, and well known to those skilled in the art.

In this embodiment, two modes of communication are possible. In the first, the server initiates an upload of the actual parameters being applied to the patient, receives these from the stimulator, and stores these in its memory, accessible to the authorized user as a dedicated content driven web page. The physician or authorized user can make alterations to the actual parameters, as available on the server, and then initiate a communication session with the stimulator device to download these parameters.

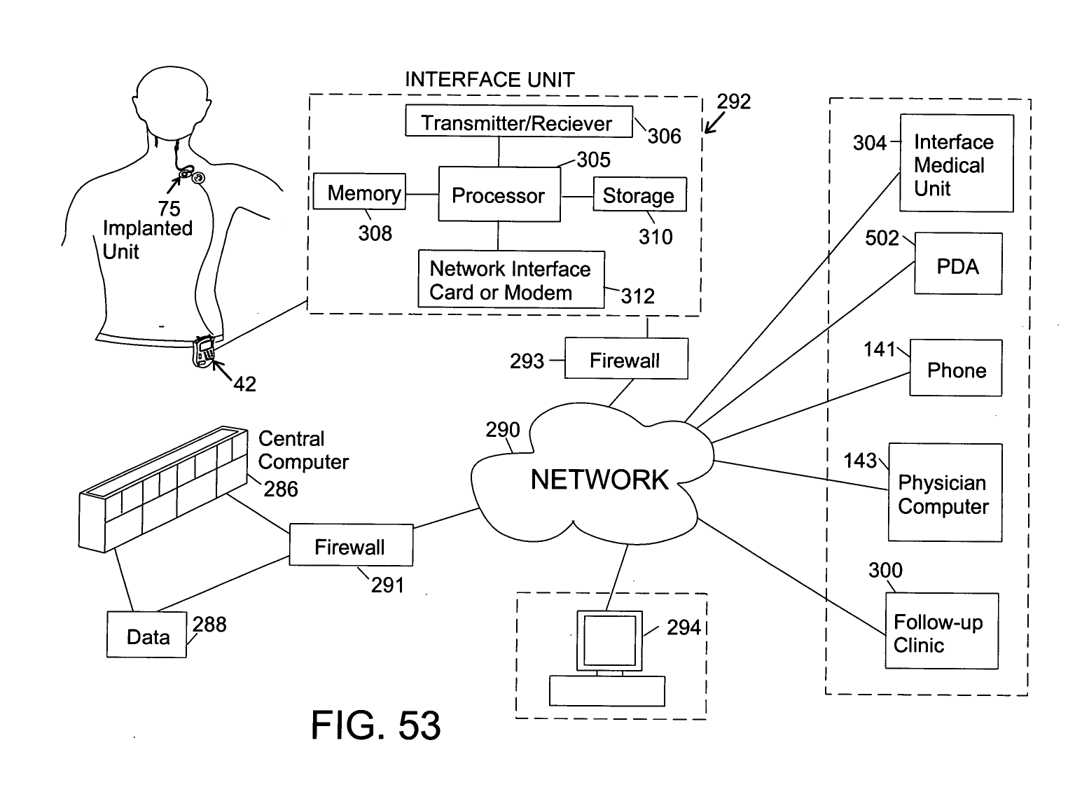

Shown in conjunction with FIG. 53, in one embodiment, the external stimulator 42 and/or the programmer 85 may also be networked to a central collaboration computer 286 as well as other devices such as a remote computer 294, PDA 502, phone 141, physician computer 143. The interface unit 292 in this embodiment communicates with the central collaborative network 290 via land-lines such as cable modem or wirelessly via the internet. A central computer 286 which has sufficient computing power and storage capability to collect and process large amounts of data, contains information regarding device history and serial number, and is in communication with the network 290. Communication over collaboration network 290 may be effected by way of a TCP/IP connection, particularly one using the internet, as well as a PSTN, DSL, cable modem, LAN, WAN or a direct dial-up connection.

The standard components of interface unit shown in block 292 are processor 305, storage 310, memory 308, transmitter/receiver 306, and a communication device such as network interface card or modem 312. In the preferred embodiment these components are embedded in the external stimulator 42 and can also be embedded in the programmer 85. These can be connected to the network 290 through appropriate security measures (Firewall) 293.

Another type of remote unit that may be accessed via central collaborative network 290 is remote computer 294. This remote computer 294 may be used by an appropriate attending physician to instruct or interact with interface unit 292, for example, instructing interface unit 292 to send instruction downloaded from central computer 286 to remote implanted unit.

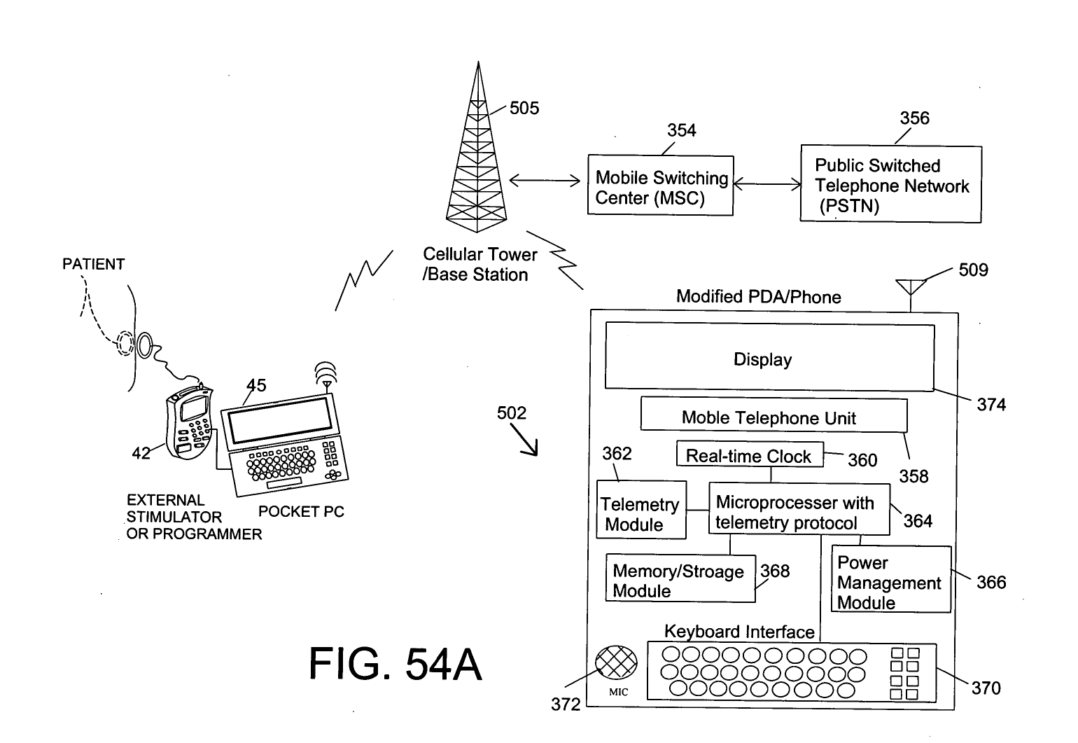

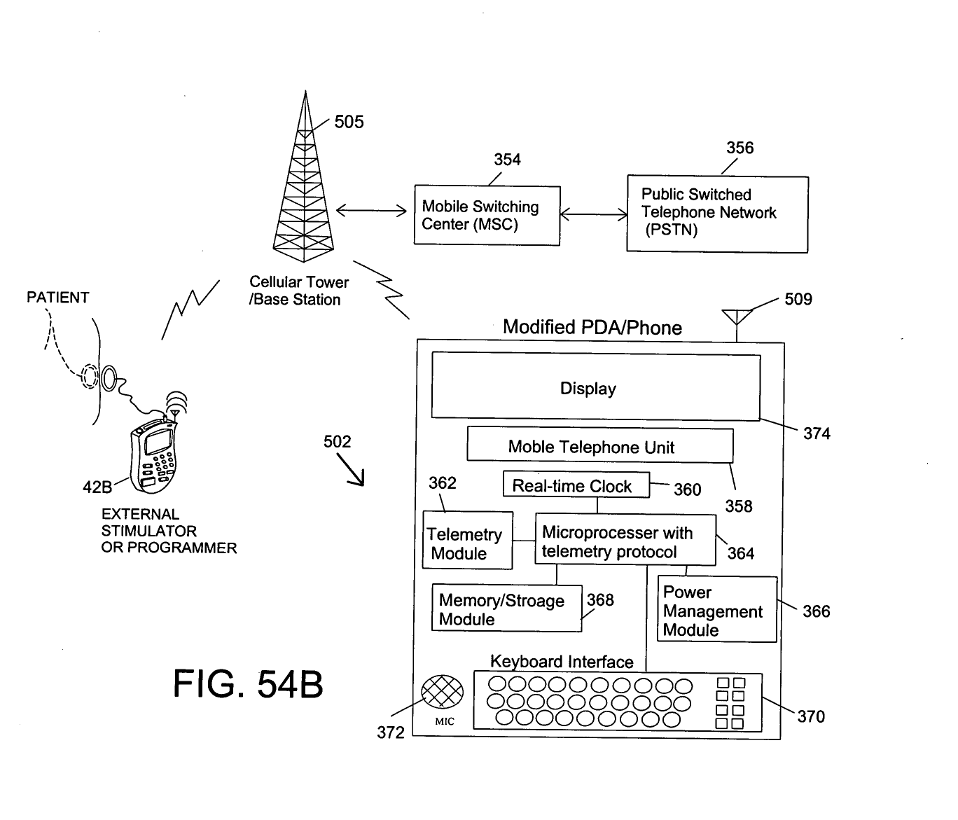

Shown in conjunction with FIGS. 54A and 54B the physician's remote communication's module is a Modified PDA/Phone 502 in this embodiment. The Modified PDA/Phone 502 is a microprocessor based device as shown in a simplified block diagram in FIGS. 65A and 65B. The PDA/Phone 502 is configured to accept PCM/CIA cards specially configured to fulfill the role of communication module 292 of the present invention. The Modified PDA/Phone 502 may operate under any of the useful software including Microsoft Window's based, Linux, Palm OS, Java OS, SYMBIAN, or the like.

The telemetry module 362 comprises an RF telemetry antenna 142 coupled to a telemetry transceiver and antenna driver circuit board which includes a telemetry transmitter and telemetry receiver. The telemetry transmitter and receiver are coupled to control circuitry and registers, operated under the control of microprocessor 364. Similarly, within stimulator a telemetry antenna 142 is coupled to a telemetry transceiver comprising RF telemetry transmitter and receiver circuit. This circuit is coupled to control circuitry and registers operated under the control of microcomputer circuit.

With reference to the telecommunications aspects of the invention, the communication and data exchange between Modified PDA/Phone 502 and external stimulator 42 operates on commercially available frequency bands. The 2.4-to-2.4853 GHz bands or 5.15 and 5.825 GHz, are the two unlicensed areas of the spectrum, and set aside for industrial, scientific, and medical (ISM) uses. Most of the technology today including this invention, use either the 2.4 or 5 GHz radio bands and spread-spectrum technology.

The telecommunications technology, especially the wireless internet technology, which this invention utilizes in one embodiment, is constantly improving and evolving at a rapid pace, due to advances in RF and chip technology as well as software development. Therefore, one of the intents of this invention is to utilize “state of the art” technology available for data communication between Modified PDA/Phone 502 and external stimulator 42. The intent of this invention is to use 3G technology for wireless communication and data exchange, even though in some cases 2.5G is being used currently.

For the system of the current invention, the use of any of the “3G” technologies for communication for the Modified PDA/Phone 502, is considered within the scope of the invention. Further, it will be evident to one of ordinary skill in the art that as future 4G systems, which will include new technologies such as improved modulation and smart antennas, can be easily incorporated into the system and method of current invention, and are also considered within the scope of the invention.

The present invention may be embodied in other specific forms without departing from the spirit or essential attributes thereof. It is therefore desired that the present embodiment be considered in all aspects as illustrative and not restrictive, reference being made to the appended claims rather than to the foregoing description to indicate the scope of the invention.

1. A method of providing electrical pulses to vagus nerve(s), and/or its branches or part thereof in a patient, and transcranial magnetic stimulation for treating or alleviating the symptoms of neurological disorders, neuropsychiatric, and cognitive impairments, comprising the steps of:
a) selecting a patient, wherein said patient is a transcranial magnetic stimulation recipient, and
b) providing electrical pulses to vagus nerve(s), and/or its branches or part thereof,
whereby, said patient receives said transcranial magnetic stimulation and vagus nerve electrical stimulation.

2. The method of claim 1, wherein said neurological and neuropsychiatric disorders and cognitive impairments further comprises at least one of depression, bipolar depression, unipolar depression, severe depression, treatment resistant depression, melancholia, mood disorders, schizophrenia, anxiety disorders, obsessive compulsive disorders, dementia including Alzheimer's disease, sleep disorders, borderline personality disorders, learning difficulties, migraines, memory impairments, and involuntary movement disorders such as in Parkinson's disease.

3. The method of claim 1, wherein said transcranial magnetic stimulation provided to said patient and said electrical pulses provided to said vagus nerve(s), and/or its branches, or parts thereof are in any sequence, any combination, or any time intervals.

4. The method of claim 1, wherein patients selected for pulsed electrical stimulation to vagus nerve have previously received transcranial magnetic stimulation therapy.

5. The method of claim 1, wherein said repetitive transcranial magnetic pulses may have a frequency between 1 Hz and 100 Hz.

6. The system of claim 1, wherein said means of providing said electric pulses to said vagus nerve(s), and/or its branches or parts thereof, further comprises at least one pulse generator from a group consisting of: a) an implanted stimulus-receiver with an external stimulator; b) an implanted stimulus-receiver comprising a high value capacitor for storing charge, used in conjunction with an external stimulator; c) a programmer-less implantable pulse generator (IPG) which is operable with a magnet; d) a microstimulator; e) a programmable implantable pulse generator; f) a combination implantable device comprising both a stimulus-receiver and a programmable IPG; g) an IPG comprising a rechargeable battery.

7. The method of claim 1, wherein said electrical pulses provided to vagus nerve(s) are provided 24 hours/day and 7 days a week in repeating ON-OFF cycles.

8. The method of claim 1, wherein said electrical pulses provided to vagus nerve(s) have predetermined parameters, which can be programmed.

9. The method of claim 1, wherein said transcranial magnetic stimulation and said electrical pulses to vagus nerve(s) are provided in addition to drug therapy.

10. A method of providing a combination of magnetic stimulation and electrical stimulation and/or nerve blocking therapy to a patient for treating, controlling, or alleviating the symptoms for at least one of depression, bipolar depression, unipolar depression, severe depression, treatment resistant depression, melancholia, schizophrenia, anxiety disorders, mood disorders, obsessive compulsive disorders, dementia including Alzheimer's disease, sleep disorders, borderline personality disorders learning difficulties, and memory impairments, comprising the steps of:
a) selecting a patient for providing said therapy;
b) providing transcranial magnetic stimulation to said patient; and
c) providing electrical pulses to vagus nerve(s), and/or its branches or part thereof in said patient.

11. The method of claim 10, wherein said transcranial magnetic stimulation provided to said patient can precede, be concurrent, or succeed said electric pulses provided to said vagus nerve(s), and/or its branches or part thereof in.

12. The method of claim 10, wherein said transcranial magnetic pulses have a frequency of about 1 kHz to 100 kHz.

13. The system of claim 10, wherein said means of providing said electric pulses to said vagus nerve(s), and/or its branches or parts thereof, further comprises at least one pulse generator from a group consisting of: a) an implanted stimulus-receiver with an external stimulator; b) an implanted stimulus-receiver comprising a high value capacitor for storing charge, used in conjunction with an external stimulator; c) a programmer-less implantable pulse generator (IPG) which is operable with a magnet; d) a microstimulator; e) a programmable implantable pulse generator; f) a combination implantable device comprising both a stimulus-receiver and a programmable IPG; g) an IPG comprising a rechargeable battery.

14. A method of modulationg the brain activity for treating or alleviating the symptoms for at least one of neurological disorders, neuropsychiatric disorders, and cognitive impairments, comprising the steps of:
a) providing a means to provide transcranial magnetic stimulation to alter the brain activity from outside the patient body, and
b) providing a means to provide electric pulses to vagus nerve(s), and/or its branches, or parts thereof, to alter the brain activity from inside the patient body.

15. The method of claim 14, wherein said transcranial magnetic stimulation provided to said patient can precede, be concurrent, or succeed said electric pulses provided to said vagus nerve(s), and/or its branches or part thereof.

16. The method of claim 14, wherein said electric pulses to said vagus nerve(s), and/or its branches or parts thereof are provided by at least one pulse generator from a group consisting of: an implanted stimulus-receiver with an external stimulator; an implanted stimulus-receiver comprising a high value capacitor for storing charge, used in conjunction with an external stimulator; a programmer-less implantable pulse generator (IPG) which is operable with a magnet; a microstimulator; a programmable implantable pulse generator; a combination implantable device comprising both a stimulus-receiver and a programmable IPG; an IPG comprising a rechargeable battery.

17. The method of claim 14, wherein said neurological and neuropsychiatric disorders and cognitive impairments further comprises at least one of depression, bipolar depression, unipolar depression, severe depression, treatment resistant depression, melancholia, mood disorders, schizophrenia, anxiety disorders, obsessive compulsive disorders, dementia including Alzheimer's disease, sleep disorders, borderline personality disorders, learning difficulties, migraines, memory impairments, and involuntary movement disorders such as in Parkinson's disease.

18. A system of providing transcranial magnetic pulses and electric pulses to the vagus nerve(s), in a patient for treating or alleviating the symptoms for at least one of neurological disorders, neuropsychiatric disorders, and cognitive impairments, comprising:
a) a means for providing transcranial magnetic pulses, wherein said means comprises a means for generating repetitive magnetic pulses, and coils for delivering said pulses to brain of said patient;
b) a means for providing electrical pulses to vagus nerve(s) in a patient, wherein said means comprises implantable and external components.

19. The method of claim 18, wherein said neurological and neuropsychiatric disorders and cognitive impairments further comprises at least one of depression, bipolar depression, unipolar depression, severe depression, treatment resistant depression, melancholia, mood disorders, schizophrenia, anxiety disorders, obsessive compulsive disorders, dementia including Alzheimer's disease, sleep disorders, borderline personality disorders, learning difficulties, migraines, memory impairments, and involuntary movement disorders such as in Parkinson's disease.

20. The system of claim 18, wherein said means to provide transcranial magnetic pulses provides pulses that have a frequency between 1 Hz to 100 Hz.

21. The system of claim 18, wherein said means of providing said electric pulses to said vagus nerve(s), and/or its branches or parts thereof, further comprises at least one pulse generator from a group consisting of: a) an implanted stimulus-receiver with an external stimulator; b) an implanted stimulus-receiver comprising a high value capacitor for storing charge, used in conjunction with an external stimulator; c) a programmer-less implantable pulse generator (IPG) which is operable with a magnet; d) a microstimulator; e) a programmable implantable pulse generator; f) a combination implantable device comprising both a stimulus-receiver and a programmable IPG; g) an IPG comprising a rechargeable battery.
